(19) United States
(12) Patent Application Publication
(10) Pub. No.: US 2026/0364181 A1
(43) Pub. Date: Nov. 19, 2026

(54) AKR1C3 Targeted Heterobifunctional Small Molecule Proteolysis Targeting Chimeras

(71) Applicants: BOARD OF REGENTS OF THE UNIVERSITY OF NEBRASKA, Lincoln, NE (US); The Trustees of the University of Pennsylvania, Philadelphia, PA (US)

(72) Inventors: Paul Trippier, Papillion, NE (US); Angelica Carmona, Omaha, NE (US); Trevor Penning, Springfield, PA (US)

(21) Appl. No.: 19/101,958

(22) PCT Filed: Aug. 9, 2023
(86) PCT No.: PCT/US2023/029842

§ 371 Date: Feb. 7, 2025

Related U.S. Application Data
(60) Provisional application No. 63/396,375, filed on Aug. 9, 2022.

(51) Int. Cl. A61K 47/55; A61K 47/54; A61P 35/00

(52) U.S. Cl. CPC A61K 47/55; A61K 47/545; A61P 35/00

(57) ABSTRACT

Provided herein are compounds of compounds, or pharmaceutically acceptable salts thereof, having the structure of Formula (0): A-L-B, wherein A is a AKR1C3 inhibitor and/or binder moiety; B is a proteolysis targeting moiety; and L is a linker moiety, as described herein. The disclosure also provides pharmaceutical compositions comprising the disclosed compounds or pharmaceutically acceptable salts thereof and a pharmaceutically acceptable carrier or excipient and further provides methods of treating, inhibiting, and/or diseases (e.g., cancer).

### STATEMENT OF U.S. GOVERNMENT SUPPORT

This invention was made with government support under R01 CA226436 awarded by the National Institutes of Health. The government has certain rights in the invention.

### BACKGROUND

Aldo-keto reductase 1 C3 (AKR1C3), also known as type 5 17β-hydroxysteroid dehydrogenase (17β-HSD), is a member of the aldo-keto reductase superfamily of proteins. It is a soluble monomeric NAD(P)(H)-dependent oxidoreductase, which catalyzes the reduction of carbonyl and alcohol groups. The protein is overexpressed in many leukemias, prostate, and other cancers. AKR1C3 plays a vital role in regulating myeloid and lymphoblast cell differentiation, proliferation, and apoptosis of hematological malignant cells; catalyzes the formation of potent androgens responsible for tumor proliferation and aggression in prostate and other hormone-dependent cancers and through its reductase activity, contributes to drug resistance against a wide variety of chemotherapeutics. The AKR1C family includes the related AKR1C1 and AKR1C2 proteins which share >84% sequence homology with AKR1C3. The requirement for selective inhibition of AKR1C3 for therapeutic effect differs depending on cancer type. While selective inhibition of AKR1C3 in castration-resistant prostate cancer is desirable for induction of tumor cell death (via suppression of androgen synthesis) and countering drug resistance, the situation in hematological malignancies is debated in the literature. All three enzymes have been reported to play a role in chemotherapy resistance in T-cell acute lymphoblastic leukemia, and a pan-AKR1C inhibitor outperformed the AKR1C3 selective inhibitor medroxyprogesterone acetate in reducing cell viability in several acute myeloid leukemia (AML) cell lines (T-ALL). Contrary to these studies, it has been shown that more potent AKR1C3 inhibitors with greater selectivity reverse drug resistance to etoposide, daunorubicin, and cytarabine in AML cells and patient-derived T-ALL cells.

### SUMMARY

There is a need for compounds that are selective inhibitors of AKR1C3 to treat conditions associated with aberrant AKR1C3 activity (e.g., cancer).

The disclosure provides compounds, or pharmaceutically acceptable salts thereof, having the structure of Formula (0): A-L-B, wherein A is a AKR1C3 inhibitor and/or binder moiety; B is a proteolysis targeting moiety; and L is a linker moiety.

The disclosure also provides pharmaceutical compositions comprising the disclosed compounds or pharmaceutically acceptable salts thereof and a pharmaceutically acceptable carrier or excipient. The disclosure further provides methods of treating, inhibiting, and/or diseases in a subject in need thereof, wherein the method comprises administering to the subject a therapeutically effective amount of the disclosed compounds or pharmaceutically acceptable salts thereof.

### BRIEF DESCRIPTION OF THE DRAWINGS

FIG. 1 shows the chemical structures of representative active and selective AKR1C3 inhibitors (Compounds 1-3), Compound 4, and Compound IA with warhead, PEG2 linker, and E3-ligase lenalidomide.

FIG. 2A shows Western blots and quantification of AKR1C3, AKR1C1/C2 and ARv7 protein expression in 22Rv1 prostate cancer cells treated with AKR1C3 inhibitor 3 at 43 nM.

FIG. 2B shows Western blots and quantification of AKR1C3, AKR1C1/C2 and ARv7 protein expression in 22Rv1 prostate cancer cells treated with Compound 3 at 1 μM.

FIG. 2C shows Western blots and quantification of AKR1C3, AKR1C1/C2 and ARv7 protein expression in 22Rv1 prostate cancer cells treated with Compound 4 at 1 μM.

FIG. 2D shows Western blots and quantification of AKR1C3, AKR1C1/C2 and ARv7 protein expression in 22Rv1 prostate cancer cells treated with and D) DMSO; for 0, 24, 48, and 72 hours. Images representative of two separate experiments.

FIG. 3 shows docking predictions of Compound IA (PROTAC) binding to AKR1C3. The PROTAC warhead (gold) is predicted to bind into the same SP1 pocket of AKR1C3 (PDB ID: 3UG8) as the known inhibitor indomethacin (green, overlay). A PEG2 linker with a triazole anchor point predicts sufficient length to engender solvent exposure of the E3 ligase ligand.

FIG. 4A shows the activity of compound IA (“PROTAC 5”) to ameliorate 22Rv1 prostate cancer cell viability upon treatment for 72 h. IC₅₀ was obtained by MTS assay. Data is representative of the mean±standard deviation of two independent experiments performed in duplicate.

FIG. 4B shows the activity of compound 3 to ameliorate 22Rv1 prostate cancer cell viability upon treatment for 72 h.

FIG. 4C shows the activity of compound 6 (E3 ligase ligand lenalidomide) to ameliorate 22Rv1 prostate cancer cell viability upon treatment for 72 h.

FIG. 5A shows the Western blot analyses of AKR1C3 degradation upon the treatment of lenalidomide 6 (10 μM) and Compound IA (“PROTAC 5”) at different concentrations (1, 10, and 50 μM) for 24 h.

FIG. 5B shows the quantitative analyses of the relative AKR1C3 protein levels at 1, 10, and 50 μM from the blot in FIG. 5A.

FIG. 5C shows the quantification of the relative mean AKR1C3 protein levels at 1, 10, and 50 μM from the blot in FIG. 5A.

FIG. 5D shows the Western blot analyses of AKR1C3 degradation upon the treatment of lenalidomide 6 (1 μM) and 5 at different concentrations (0.5, 1, 10, 50, 100, 250 and 500 nM) for 24 h.

FIG. 5E shows the quantitative analyses of the relative AKR1C3 protein levels at 0.5, 1, 10, 50, 100, 250, and 500 nM from the blot in FIG. 5D.

FIG. 5F shows the quantification of the relative mean AKR1C3 protein levels at 0.5, 1, 10, 50, 100, 250, and 500 nM from the blot in FIG. 5D. Data was obtained from two independent experiments and shown as mean±standard deviation.

FIG. 6A shows the time study of degradation effect of AKR1C3 upon treatment of 5 (1 nM) at different time points (0, 2, 4, 6, 12, 16, 24, 48, and 72 h). Blots are representative of two separate experiments.

FIG. 6B shows the quantitative analysis for AKR1C3 expression from the blot in FIG. 6A.

FIG. 6C shows the combined quantification of AKR1C3 expression from n=2 experiments; Data obtained from two biological independent experiments are depicted as mean±standard deviation.

FIG. 7A shows results from a time study of degradation of AKR1C3, AKR1C1/C2, and ARv7 upon treatment of Compound IA (“PROTAC 5”) (10 nM) at different time points (0, 2, 4, 6, 12, 16, 24, 48, and 72 h). Blots are representative of two separate experiments.

FIG. 7B shows the combined quantification for AKR1C3 and AKR1C1/C2 expression from the blot in FIG. 7A.

FIG. 7C shows the quantification for AKR1C3 expression, AKR1C1/C2 expression, and ARv7 expression from the blot in FIG. 7A. Data obtained from two biological independent experiments is the mean±standard deviation. *p<0.05 by two-tailed, unpaired Mann-Whitney test.

FIG. 8A shows a Western blot of AKR1C3 expression and the relative AKR1C3 expression after 72 hour treatment of Compound 5 versus small molecule AKR1C3 inhibitors 3 and 4 at 10 nM concentration.

FIG. 8B shows a representative Western blot of concentration-dependent protein degradation after treatment of Compound 5 for 72 hours.

FIG. 8C shows DC₅₀ calculation of AKR1C3 degradation.

FIG. 8D shows DC₅₀ calculation of AKR1C1/C2 degradation.

FIG. 8E shows DC₅₀ calculation of ARv7 degradation.

FIG. 8F shows the effect on protein degradation with 2 hour pretreatment of DMSO or pretreatment with proteasome inhibitor MG132 (3 μM). Cells were then treated with Compound IA at 10 nM.

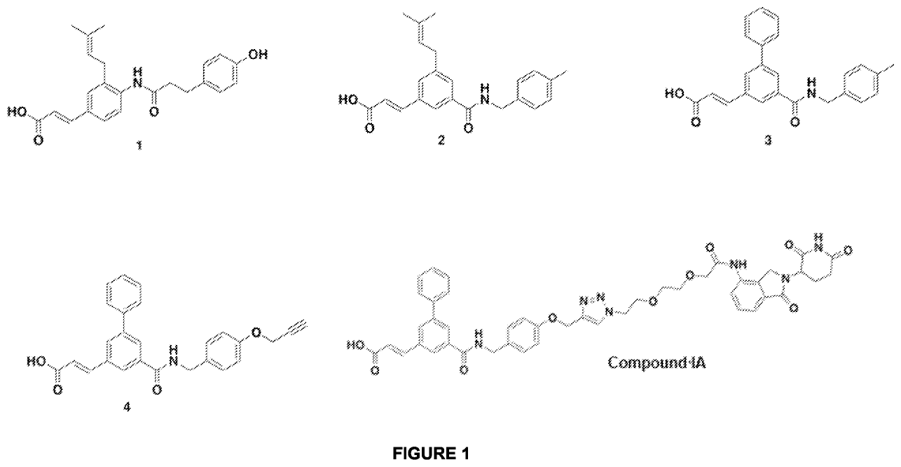

### DETAILED DESCRIPTION

The present disclosure provides compounds useful for treating, inhibiting, and/or preventing diseases associated with aberrant AKR1C3 activity.

The compounds and pharmaceutical salts disclosed herein can provide several advantages over conventional compounds and treatment regimens. Proteolysis-targeting chimeras (PROTACs) are heterobifunctional molecules consisting of a ligand that binds to the protein of interest connected by a linker to a ligand that binds to and recruits the E3 ubiquitin ligase. Once the target protein and either a component of E3 ubiquitin ligase or the E2 ligase are bound, a ternary complex is formed, followed by polyubiquitination and subsequent degradation of the target protein by the 26S proteasome; the PROTAC is then recycled to carry out successive rounds of polyubiquitination. A PROTAC degradation strategy possesses several advantages over small molecule inhibition: the ability to target undruggable proteins, induce complete removal of the target protein to overcome drug resistance that might be mediated via mutations or protein overexpression and accumulation, and/or activity at sub-stoichiometric concentrations. The PROTAC may also have increased potency and selectivity achievable via protein-protein interactions between the protein of interest and E3 ubiquitin ligase and prolonged pharmacodynamic effects without continuous PROTAC exposure.

The disclosed compounds are suitable for the degradation of AKR1C3 with a dual effect to degrade ARv7 and AKR1C1/C2. The compounds disclosed herein inhibit or induce degradation of AKR1C3 after exposure (e.g., 4 h post exposure). AKR1C3 and ARv7 are highly implicated in the development of chemotherapeutic resistance to clinical androgen receptor (AR) antagonists in advanced prostate cancer. Without wishing to be bound to any particular theory, it is believed that degradation of AKR1C3/ARv7 axis represents a promising therapeutic strategy to counter drug resistance.

AKR1C3 and ARv7 are highly implicated in the development of chemotherapeutic resistance to clinical AR antagonists in advanced prostate cancer. Degradation of the AKR1C3/ARv7 axis represents a promising therapeutic strategy to counter drug resistance. As described herein, in some cases the disclosure provides compounds of Formula (0), including compounds of Formula (I), compound of Formula (IA), and compound of Formula (II). The disclosed compounds (e.g., Compound (IA)) degrade AKR1C3 with a dual effect to degrade ARv7 and AKR1C1/C2 and are more potent than small molecule inhibitors to induce degradation and do so from 4 hours post exposure.

### Compounds of the Disclosure

The disclosure provides compounds, or pharmaceutically acceptable salts thereof, having the structure of Formula (0): A-L-B, wherein A is an AKR1C3 inhibitor and/or binder moiety; B is a proteolysis targeting moiety; and L is a linker moiety.

AKR1C3 Inhibitor and/or Binder Moiety “A”

The compounds disclosed herein comprise an AKR1C3 inhibitor and/or binder moiety A. Suitable AKR1C3 inhibitor and/or binder moieties include a small molecule moiety, a peptide moiety, or an antibody moiety or a fragment thereof. In some cases, the AKR1C3 inhibitor and/or binder moiety is a small molecule moiety. In some cases, the AKR1C3 inhibitor and/or binder moiety is a peptide moiety. In some cases, the AKR1C3 inhibitor and/or binder moiety is an antibody moiety or a fragment thereof. In some cases, the AKR1C3 inhibitor and/or binder moiety is an antibody moiety. In some cases, the AKR1C3 inhibitor and/or binder moiety is a fragment of an antibody moiety.

In some cases, the AKR1C3 inhibitor and/or binder moiety is a small molecule moiety adapted to be coupled to linker L through an amide moiety. In some cases, the AKR1C3 inhibitor and/or binder moiety is derived from a compound comprising a carboxylic acid, which is adapted to be coupled through L through an amide moiety. In some cases, the AKR1C3 inhibitor and/or binder moiety is derived from a compound comprising an amine, which is adapted to be coupled through L through an amide moiety.

For example, the AKR1C3 is a moiety of Formula (A1):

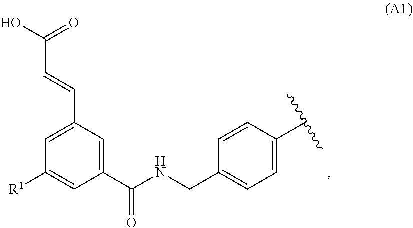

wherein R¹ is selected from C₁-C₆alkyl, C₆-C₁₀aryl, C₂-C₆alkenyl, C₁-C₆alkoxy, SO₂R^{S}, (CO)ₘN(R^{a})₂, and 5-10 membered heteroaromatic having 1-3 ring heteroatoms selected from N, O, S; R^{S} is C₁-C₆alkyl or substituted or unsubstituted C₆-C₁₀aryl; each R^{a} independently is H or C₁-C₆alkyl; m is 0 or 1; and R¹ is optionally substituted.

In some cases, R¹ can be further substituted. For example, R¹ is optionally substituted with one or more substituents selected from C₆-C₁₀aryl; 5-15 membered heteroaryl having 1-4 ring heteroatoms selected from N, O, and S; formyl; C₃-C₆cycloalkyl; halo; cycloheteroalkyl-alkylene; hydroxy-C₁-C₃alkylene; methoxymethyl; phenyoxy; cyano; C₁-C₆alkyl; C₁-C₆haloalkyl; and 5-7 membered fused cycloheteroalkyl having 1 to 3 ring heteroatoms selected from N, O, and S. The R¹ moiety can be further substituted with one or more substituents selected from C₆-C₁₀aryl; 5-15 membered heteroaryl having 1-4 ring heteroatoms selected from N, O, and S; formyl; C₃-C₆cycloalkyl; halo; cycloheteroalkyl-alkylene; hydroxy-C₁-C₃alkylene; methoxymethyl; phenyoxy; cyano; C₁-C₆alkyl; C₁-C₆haloalkyl; and 5-7 membered fused cycloheteroalkyl having 1 to 3 ring heteroatoms selected from N, O, and S. In R¹ can be further substituted with one or more substituents selected from C₆-C₁₀aryl, C₆-C₁₀aryloxy, and halo. For example, R¹ can be further substituted with halo.

In some cases, R¹ is C₁-C₆alkyl. In some cases, R¹ is C₆-C₁₀aryl (e.g., phenyl). In some cases, R¹ is C₂-C₆alkenyl. In some cases, R¹ is C₁-C₆alkoxy. In some cases, R¹ is SO₂R^{S}, wherein R^{S} is C₁-C₆alkyl or substituted or unsubstituted C₆-C₁₀aryl. In some cases, R¹ is (CO)ₘN(R^{a})₂, wherein each R^{a} independently is H or C₁-C₆alkyl. In some cases, R¹ is 5-10 membered heteroaromatic having 1-3 ring heteroatoms selected from N, O, S

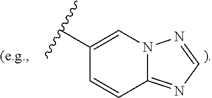 [SMILES: *c1ccc2ncnn2c1]

In some cases, m is 0. In some cases, m is 1.

In some cases, the AKR1C3 inhibitor and/or binder moiety is selected from

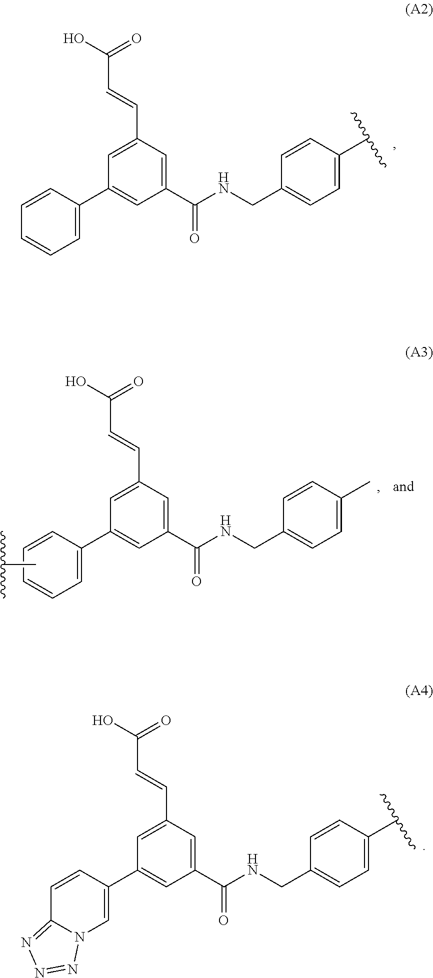

### Linker Moiety “L”

The compounds disclosed herein comprise a linker moiety L. In some cases, the linker comprises one or more optionally substituted alkyl group, an ester, an amine, amide, a urea, a sulfide, a thiol ester, a thiol, and a combination thereof. Suitable substituents for the alkyl group include, for example, ester groups, thiol groups, amine groups, C6-C₁₂aryl groups (e.g. phenyl), benzyl groups, heterocyclic groups (e.g., piperazine groups), heteroaromatic groups. In some cases, the linker can comprise between 1 and 30 carbon atoms, wherein the carbon atoms may be substituted with nitrogen, oxygen, and other groups such as phenyl, benzyl, and piperazine.

In some cases, the linker moiety is C₁-C₁₈alkylene interrupted with one or more of (i) non-adjacent heteroatom(s) selected from O, S, and NR^{N}, (ii) C₆-C₁₀arylene, (iii) 5-10 membered cycloheteroalkylene having 1-3 ring heteroatoms selected from N, O, and S, and (iv) C(O)NR^{N}, and (v) NR^{N}C(O); and each R^{N} is independently H or C₁₋₆ alkyl.

In some cases, the linker moiety is C₁-C₁₈alkylene interrupted with one or more non-adjacent heteroatom(s) selected from O, S, and NR^{N}, wherein each R^{N} is independently H or C₁₋₆ alkyl. In some cases, the linker moiety is C₁-C₁₈alkylene interrupted with one or more of C₆-C₁₀arylene. In some cases, the linker moiety is C₁-C₁₈alkylene interrupted with one or more 5-10 membered cycloheteroalkylene having 1-3 ring heteroatoms selected from N, O, and S. In some cases, the linker moiety is C₁-C₁₈alkylene interrupted with one or more C(O)NR^{N}, wherein each R^{N} is independently H or C₁₋₆ alkyl. In some cases, the linker moiety is C₁-C₁₈alkylene interrupted with one or more NR^{N}C(O), wherein each R^{N} is independently H or C₁₋₆ alkyl.

In some cases, the linker moiety is selected from

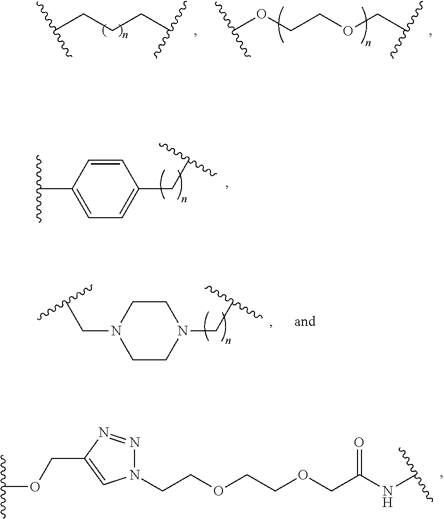

wherein n is 0-10.

In some cases, n is 0, 1, 2, 3, 4, 5, 6, 7, 8, 9, or 10.

In some cases, the linker is

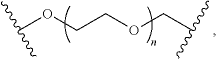

for example,

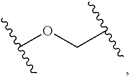 [SMILES: *CO*]

In some cases, the linker is

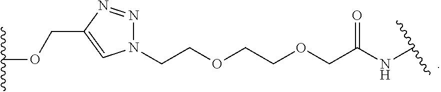 [SMILES: *NC(=O)COCCOCCn1cc(CO*)nn1]

### Proteolysis Targeting Moiety “B”

The compounds disclosed herein comprise a proteolysis targeting moiety “B”.

The proteolysis targeting moiety may be selected without limitation from a small molecule moiety, a peptide moiety, an electrophilic moiety, and an antibody moiety or a fragment thereof. In some cases, the proteolysis targeting moiety is a small molecule moiety. In some cases, the proteolysis targeting moiety is a peptide moiety. In some cases, the proteolysis targeting moiety is an electrophilic moiety. In some cases, the proteolysis targeting moiety is an antibody moiety or a fragment thereof.

In some cases, the proteolysis targeting moiety is an electrophilic moiety. An illustrative example of an electrophilic moiety is a terminal alkyne (e.g., ethynyl).

In some cases, the proteolysis targeting moiety is an E3 ligase binding moiety. The E3 ligase binding moiety can be selected from a small molecule moiety, a peptide moiety, and an antibody moiety or fragment thereof. In some cases, the E3 ligase binding moiety includes, but is not limited to, an IκBα-derived motif (such as a phosphopeptide motif), a HIF-1α derived motif (such as a HIF-1α pentapeptide or octopeptide motif), nutlin, bestatin, methyl-bestatin, thalidomide, pomalidomide, lenalidomide, thalidomide analogs, VHL binding molecules (including those described in Galdeano et al, J. Med Chem 2014, 57 (20): 8657-8663) which is herein incorporated by reference). The E3 ligase binding moieties are capable of binding to and/or recruiting an E3 ligase including but not limited to VHL, cereblon, MDM2, clAP1, and APC/C^{CDH-1}. In some cases, the E3 ligase binding molecule binds to and/or recruits the E3 ligase cereblon.

In some cases, the E3 ligase binding moiety has a structure of formula:

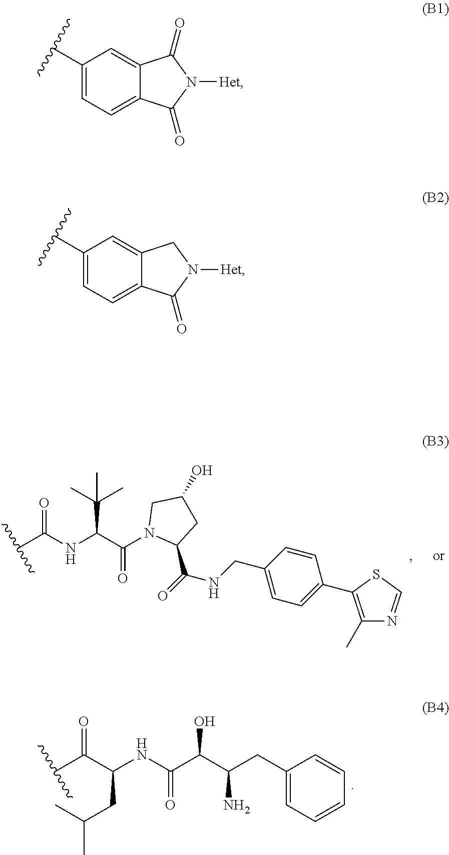

wherein Het is a 3-7 membered heterocycloalkyl having 1-3 ring heteroatoms selected from O, S, and N, and said heterocycloalkyl is optionally substituted with one or more oxo.

In some cases, the E3 ligase binding moiety has a structure of formula:

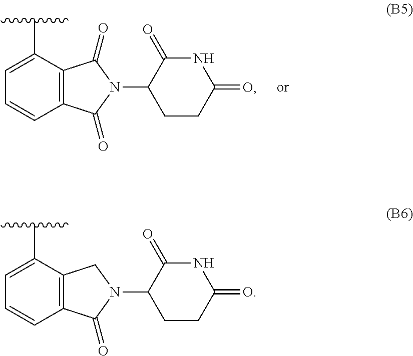 [SMILES: *c1cccc2c1C(=O)N(C1CCC(=O)NC1=O)C2=O ; *c1cccc2c1CN(C1CCC(=O)NC1=O)C2=O]

In some cases, the E3 ligase binding moiety has a structure of formula (B6).

In some cases, the disclosure provides compounds of Formula (I), or a pharmaceutical salt thereof:

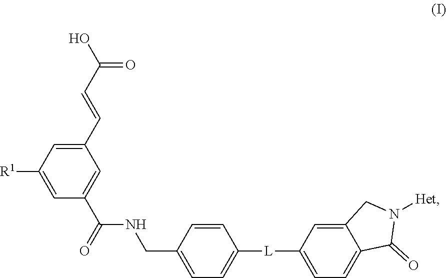

wherein R¹, L, and Het are as described herein. In some cases, R¹ of Formula (I) is phenyl. In some cases, R¹ of Formula (I) is

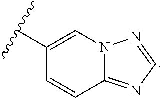 [SMILES: *c1ccc2ncnn2c1]

In some cases, the disclosure provides a compound of Formula (IA):

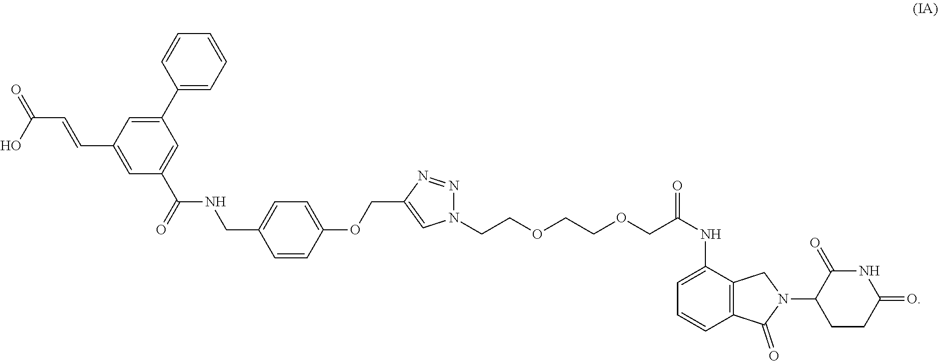 [SMILES: O=C(O)/C=C/c1cc(C(=O)NCc2ccc(OCc3cn(CCOCCOCC(=O)Nc4cccc5c4CN(C4CCC(=O)NC4=O)C5=O)nn3)cc2)cc(-c2ccccc2)c1]

compound of Formula (IA) is also referred to herein as “PROTAC 5” or “Compound 5” or “(5)” or “5”.

In some cases, the disclosure provides a compound of Formula (II):

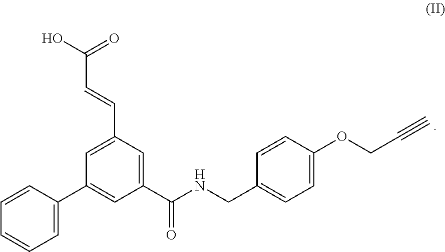 [SMILES: C#CCOc1ccc(CNC(=O)c2cc(/C=C/C(=O)O)cc(-c3ccccc3)c2)cc1]

The compounds disclosed herein can be in the form of a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N,N [symbol] dibenzylethylenediamine, 2-hydroxyethylamine, bis-(2-hydroxyethyl)amine, tri-(2-hydroxyethyl)amine, procaine, dibenzylpiperidine, dehydroabietylamine, N,N [symbol] bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### DEFINITIONS

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₄ alkyl refers to an alkyl group that has 4 carbon atoms. C₁-C₈ alkyl refers to an alkyl group having a number of carbon atoms encompassing the entire range (e.g., 1 to 8 carbon atoms), as well as all subgroups (e.g., 1-8, 2-8, 3-8, 4-8, 5-8, 6-8, 7-8, 1, 2, 3, 4, 5, 6, 7, and 8 carbon atoms). Nonlimiting examples of alkyl groups include, methyl, ethyl, n-propyl, isopropyl, n-butyl, sec-butyl(2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group.

The term “alkylene” used herein refers to an alkyl group having a substituent. For example, the term “alkylenehalo” refers to an alkyl group substituted with a halo group. For example, an alkylene group can be —CH₂CH₂— or —CH₂—. The term Cₙ means the alkylene group has “n” carbon atoms. For example, C₁₋₁₈ alkylene refers to an alkylene group having a number of carbon atoms encompassing the entire range, as well as all subgroups, as previously described for “alkyl” groups. Unless otherwise indicated, an alkylene group can be an unsubstituted alkylene group or a substituted alkylene group.

The term “alkenyl” used herein refers to an unsaturated aliphatic group analogous in length and possible substitution to an alkyl group described above, but that contains at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. For example, a straight chain or branched alkenyl group can have six or fewer carbon atoms in its backbone (e.g., C₂-C₆ for straight chain, C₃-C₆ for branched chain). The term “C₂-C₆” includes chains having a number of carbon atoms encompassing the entire range (e.g., 2 to 6 carbon atoms), as well as all subgroups (e.g., 2-6, 2-5, 2-4, 3-6, 2, 3, 4, 5, and 6 carbon atoms). The term “C₃-C₆” includes chains having a number of carbon atoms encompassing the entire range (e.g., 3 to 6 carbon atoms), as well as all subgroups (e.g., 3-6, 3-5, 3-4, 3, 4, 5, and 6 carbon atoms). Unless otherwise indicated, an alkenyl group can be an unsubstituted alkenyl group or a substituted alkenyl group.

The term “alkenylene” used herein refers to an alkenyl group having a substituent. For example, the term “alkenylenehalo” refers to an alkyl group substituted with a halo group. For example, an alkylene group can be —CH═CH—. The term Cₙ means the alkenylene group has “n” carbon atoms. For example, C₂₋₆ alkenylene refers to an alkenylene group having a number of carbon atoms encompassing the entire range, as well as all subgroups, as previously described for “alkenyl” groups. Unless otherwise indicated, an alkenylene group can be an unsubstituted alkenylene group or a substituted alkenylene group.

As used herein, an alkylene which is “interrupted” is understood to be an alkylene group in which at one or more (e.g., 1-5, 1-4, 1-3, 1-2, 1, 2, 3, 4, or 5) positions on the alkylene chain is inserted a group selected from one or more of (i) non-adjacent heteroatom(s) selected from O, S, and NR^{N}, (ii) C(O)NR^{N}, and (iii) NR^{N}C(O). The interruptions can be consecutive for various combinations of these interrupting groups (e.g., a heteroatom next to a C(O)NR^{N} moiety), except that two heteroatoms cannot be adjacent or consecutive to each other.

As used herein an alkylene which is interrupted with “one or more” groups is understood to be interrupted with from 1 to n-1 groups, wherein n is the number of carbon atoms in the alkylene chain. For example, a C₆-alkylene which is optionally interrupted with one or more groups can be interrupted with one, two, three, four, or five groups.

The term “alkynyl” used herein refers to an unsaturated aliphatic group analogous in length and possible substitution to an alkyl group described above, but that contains at least one triple bond. For example, the term “alkynyl” includes straight chain alkynyl groups (e.g., ethynyl, propynyl, butynyl, pentynyl, hexynyl, heptynyl, octynyl), and branched alkynyl groups. For example, a straight chain or branched alkynyl group can have eight or fewer carbon atoms in its backbone (e.g., C₂-C₈ for straight chain, C₄-C₈ for branched chain). The term “C₂-C₈” includes chains having a number of carbon atoms encompassing the entire range (e.g., 2 to 8 carbon atoms), as well as all subgroups (e.g., 2-6, 2-5, 2-4, 3-6, 2, 3, 4, 5, and 6 carbon atoms). The term “C₄-C₈” includes chains having a number of carbon atoms encompassing the entire range (e.g., 4 to 8 carbon atoms), as well as all subgroups (e.g., 4-6, 4-5, 4, 5, and 6 carbon atoms). Unless otherwise indicated, an alkynyl group can be an unsubstituted alkynyl group or a substituted alkynyl group.

The term “alkynylene” used herein refers to an alkynyl group having a substituent. For example, an alkylene group can be

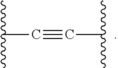 [SMILES: *C#C*]

The term Cₙ means the alkynylene group has “n” carbon atoms. For example, C₂₋₈ alkynylene refers to an alkynylene group having a number of carbon atoms encompassing the entire range, as well as all subgroups, as previously described for “alkynyl” groups. Unless otherwise indicated, an alkynylene group can be an unsubstituted alkynylene group or a substituted alkynylene group.

As used herein, the term “cycloalkyl” refers to an aliphatic cyclic hydrocarbon group containing three to eleven carbon atoms (e.g., 3, 4, 5, 6, 7, 8, 9, 10, or 11 carbon atoms). The term Cₙ means the cycloalkyl group has “n” carbon atoms. For example, C₅ cycloalkyl refers to a cycloalkyl group that has 5 carbon atoms in the ring. C₆-C₁₁ cycloalkyl refers to cycloalkyl groups having a number of carbon atoms encompassing the entire range (e.g., 6 to 11 carbon atoms), as well as all subgroups (e.g., 6-7, 6-8, 7-8, 6-9, 6, 7, 8, 9, 10, and 11 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group. When a cycloalkyl group is fused to another cycloalkyl group, then each of the cycloalkyl groups can contain three to twelve carbon atoms unless specified otherwise. Unless otherwise indicated, a cycloalkyl group can be unsubstituted or substituted.

The term “cycloalkenyl” is defined similarly as “cycloalkyl” except that the ring comprises at least one double bond, without being aromatic. The cycloalkenyl groups described herein can be isolated or fused to another cycloalkenyl group. Unless otherwise indicated, a cycloalkenyl group can be unsubstituted or substituted.

As used herein, the term “heterocycloalkyl” is defined similarly as cycloalkyl, except the ring contains one to three heteroatoms independently selected from oxygen, nitrogen, and sulfur. In particular, the term “heterocycloalkyl” refers to a ring containing a total of three to eleven atoms (e.g., three to seven, or five to eleven), of which 1, 2, 3 or three of those atoms are heteroatoms independently selected from the group consisting of oxygen, nitrogen, and sulfur, and the remaining atoms in the ring are carbon atoms. Nonlimiting examples of heterocycloalkyl groups include piperdine, pyrazolidine, tetrahydrofuran, tetrahydropyran, dihydrofuran, morpholine, and the like. The heterocycloalkyl groups described herein can be isolated or fused to another heterocycloalkyl group. Heterocycloalkyl groups can be saturated or partially unsaturated ring systems. Unless otherwise indicated, a heterocycloalkyl group can be unsubstituted or substituted.

As used herein, the term “aryl” refers to a monocyclic aromatic hydrocarbon group, such as phenyl or a bicyclic aromatic hydrocarbon group, such as naphthyl. Unless otherwise indicated, an aryl group can be unsubstituted or substituted with one or more groups. Aryl groups can be isolated (e.g., phenyl) or fused to another aryl group (e.g., naphthyl, anthracenyl), a cycloalkyl group (e.g. tetraydronaphthyl), a heterocycloalkyl group, and/or a heteroaryl group. Exemplary aryl groups include, but are not limited to, phenyl, chlorophenyl, methylphenyl, methoxyphenyl, trifluoromethylphenyl, nitrophenyl, 2,4-methoxychlorophenyl, and the like. Throughout, the abbreviation “Ph” refers to phenyl and “Bn” refers to benzyl (i.e., CH₂phenyl).

As used herein, the term “heteroaryl” refers to a monocyclic or bicyclic aromatic ring having 5 to 14 total ring atoms, and containing one to three heteroatoms selected from nitrogen, oxygen, and sulfur atom in the aromatic ring. Unless otherwise indicated, a heteroaryl group can be unsubstituted or substituted. Examples of heteroaryl groups include, but are not limited to, thienyl, furyl, pyridyl, pyrrolyl, oxazolyl, triazinyl, triazolyl, isothiazolyl, isoxazolyl, imidazolyl, pyrazinyl, pyrimidinyl, thiazolyl, and thiadiazolyl.

As used herein, the term “oxo” refers to a ═O group.

As used herein, the term “halo” refers to a F (fluoro), Cl (chloro), Br (bromo), or I (iodo) group.

As used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl, heterocycloalkyl, aryl, heteroaryl, hydroxyl, oxy, alkoxy, heteroalkoxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

As used herein, the phrase “optionally substituted” means unsubstituted (e.g., substituted with a H) or substituted. As used herein, the term “substituted” means that a hydrogen atom is removed and replaced by a substituent. It is understood that substitution at a given atom is limited by valency. The use of a substituent (radical) prefix name such as alkyl without the modifier “optionally substituted” or “substituted” is understood to mean that the particular substituent is unsubstituted.

As used herein, the term “therapeutically effective amount” means an amount of a compound or combination of therapeutically active compounds (e.g., a AKR1C3 degrader or combination of AKR1C3 degrader) that ameliorates, attenuates or eliminates one or more symptoms of a particular disease or condition (e.g., cancer), or prevents or delays the onset of one of more symptoms of a particular disease or condition.

As used herein, the terms “patient” and “subject” may be used interchangeably and mean animals, such as dogs, cats, cows, horses, and sheep (e.g., non-human animals) and humans. Particular patients or subjects are mammals (e.g., humans). The terms patient and subject include males and females.

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

As used herein the terms “treating”, “treat” or “treatment” and the like include preventative (e.g., prophylactic) and palliative treatment.

As used herein, the term “excipient” means any pharmaceutically acceptable additive, carrier, diluent, adjuvant, or other ingredient, other than the active pharmaceutical ingredient (API).

### Pharmaceutical Formulations, Dosing, and Routes of Administration

Further provided are pharmaceutical formulations comprising a compound as described herein (e.g., compounds of Formula 0, Formula I, Formula IA, the compounds of Table 1, or pharmaceutically acceptable salts of the compounds) and a pharmaceutically acceptable excipient.

The compounds described herein can be administered to a subject in a therapeutically effective amount (e.g., in an amount sufficient to prevent or relieve the symptoms of a disorder capable of being modulated by CDK degradation). The compounds can be administered alone or as part of a pharmaceutically acceptable composition or formulation. In addition, the compounds can be administered all at once, multiple times, or delivered substantially uniformly over a period of time. It is also noted that the dose of the compound can be varied over time.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to effect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Purely by way of illustration, the method comprises administering, e.g., from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition and type of pain, and may last one day to several months.

Suitable methods of administering a physiologically-acceptable composition, such as a pharmaceutical composition comprising the compounds disclosed herein (e.g., compounds of compounds of Formula 0, Formula I, Formula IA, the compounds of Table 1, or pharmaceutically acceptable salts of the compounds), are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent). The particular carrier employed is limited only by chemico-physical considerations, such as solubility and lack of reactivity with the compound, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The compositions used in the methods of the invention may be formulated in micelles or liposomes. Such formulations include sterically stabilized micelles or liposomes and sterically stabilized mixed micelles or liposomes. Such formulations can facilitate intracellular delivery, since lipid bilayers of liposomes and micelles are known to fuse with the plasma membrane of cells and deliver entrapped contents into the intracellular compartment.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, PA, pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

It will be appreciated that the pharmaceutical compositions and treatment methods of the invention are useful in fields of human medicine and veterinary medicine. Thus the subject to be treated is in one aspect a mammal. In another aspect, the mammal is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Methods of Use

The compounds disclosed herein are particularly advantageous for the treatment of diseases or disorders caused by aberrant expression or activity of a AKR1C3. The incidence and/or intensity of diseases or disorders associated with aberrant expression or activity of a AKR1C3 is reduced. Selective AKR1C3 inhibitors or degraders can be used for cancer prevention and treatment. The relationship between AKR1C3 activity and cancer has been examined in various studies. AKR1C3 has been found to be upregulated in prostate, breast, ovarian, certain hematological malignancies, and other cancers where it contributes to proliferation and chemotherapeutic resistance. Androgen receptor splice variant 7 (ARv7) is the most common mutation of the AR receptor that confers resistance to clinical androgen receptor antagonists in prostate cancer. AKR1C3 interacts with ARv7 promoting stabilization.

The disclosure provides methods of using the disclosed compounds. A method of inhibiting an aldo-keto reductase family 1 member C3 (AKR1C3) can include contacting the AKR1C3 with a compound of Formula (0): A-L-B, wherein A is a AKR1C3 inhibitor and/or binder moiety, B is a proteolysis targeting moiety, and L is a linker moiety, as described herein. In some cases, the disclosed methods comprise contacting AKR1C3 with a compound of Formula (I). In some cases, the disclosed methods comprise contacting AKR1C3 with a compound of Formula (IA). In some cases, the disclosed methods comprise contacting AKR1C3 with a compound of Formula (II).

Methods of degrading a AKR1C3 can include administering to a subject in need thereof a therapeutically effective amount of a compound disclosed herein, or a pharmaceutical salt thereof. The disclosure also provides methods of treating or preventing diseases or disorders comprising administering a therapeutically effective amount of a compound disclosed herein, or a pharmaceutical salt thereof. The disclosure also provides methods of treating or preventing diseases or disorders comprising administering a therapeutically effective amount of a composition disclosed herein.

In some cases, the disclosure provides methods of degrading a AKR1C3 comprising administering to a subject in need thereof a therapeutically effective amount of a composition comprising a AKR1C3 inhibitor and/or binder moiety linked to an electrophilic moiety (e.g., a terminal alkyne). In some cases, the disclosure provides methods of degrading a AKR1C3 comprising administering to a subject in need thereof a therapeutically effective amount of a composition comprising a AKR1C3 inhibitor and/or binder moiety linked to an E3 ligase binding moiety. Also provided are methods of treating or preventing a disease or disorder capable of being modulated by AKR1C3 degradation or by androgen receptor splice variant (ARv7), comprising administering to a subject in need thereof a therapeutically effective amount of a composition comprising an AKR1C3 inhibitor moiety linked to an E3 ligase binding moiety. Also provided are methods of treating or preventing a disease or disorder capable of being modulated by AKR1C3 degradation or by androgen receptor splice variant (ARv7), comprising administering to a subject in need thereof a therapeutically effective amount of a composition comprising an AKR1C3 inhibitor moiety linked to an electrophilic moiety (e.g., an alkyne).

As described herein, any of the disclosed methods can comprise administering (or contacting) one or more the compounds disclosed herein. In some cases, the disclosed methods comprise administering one or more of the compounds disclosed herein in combination with one or more additional active agent (e.g., additional anti-cancer drug).

In some cases, the disease or disorder capable of being modulated by AKR1C3 degradation or by androgen receptor splice variant (ARv7) is selected from cancer, inflammatory diseases (e.g., asthma and atopic dermatitis), and neurological diseases. In some cases, the disease or disorder is cancer, endometriosis, or multiple sclerosis.

In some cases, the disease or disorder is cancer. For example, the cancers include leukemia, lymphoma, multiple myeloma, breast cancer, prostate cancer, pancreatic cancer, colon cancer, thyroid cancer, bladder cancer, liver cancer, neuroblastoma, brain cancer, lung cancer, ovarian cancer, stomach cancer, skin cancer, cervical cancer, testicular cancer, kidney cancer, carcinoid tumors, and bone cancer. In some cases, the cancer is prostate cancer.

In some cases, the cancer is lymphoma (e.g., Hodgkin lymphoma or non-Hodgkin lymphoma). In some cases, the cancer is brain cancer (e.g., a glioma, a meningioma, or a pituitary adenoma). In some cases, the skin cancer is melanoma. In some cases, the leukemia is acute acute lymphoblastic leukemia, acute myeloid leukemia, chronic lymphocytic leukemia, T-cell acute lymphoblastic leukemia, chronic myelogenous leukemia.

The disclosure also provides compositions comprising the compounds disclosed herein. In some cases, the disclosure provides pharmaceutical compositions comprising a compound disclosed herein (or a pharmaceutically acceptable salt thereof) and a pharmaceutically acceptable carrier or excipient. In some cases, the pharmaceutical compositions disclosed herein comprise a compound of Formula (I). In some cases, the pharmaceutical compositions disclosed herein comprise a compound of Formula (IA). In some cases, the pharmaceutical compositions disclosed herein comprise a compound of Formula (II).

Also provided herein are compositions comprising a AKR1C3 binder/inhibitor moiety linked to an E3 ligase binding moiety for use in a method of degrading AKR1C3. Also provided herein are compositions comprising a AKR1C3 binder/inhibitor moiety linked to an electrophilic moiety (e.g., a terminal alkyne). for use in a method of inhibiting AKR1C3. Also provided are compositions comprising a AKR1C3 inhibitor and/or binder moiety linked to an E3 ligase binding moiety for use in a method of treating or preventing a disease or disorder capable of being modulated by AKR1C3 inhibition or degradation. Also provided are compositions comprising a AKR1C3 inhibitor and/or binder moiety linked to an electrophilic moiety (e.g., a terminal alkyne) for use in a method of treating or preventing a disease or disorder capable of being modulated by AKR1C3 inhibition or degradation.

The disclosed methods include methods for treating disease or disorder capable of being modulated by degradation of AKR1C3, e.g., cancer, comprising administering to a subject a compound that degrades a AKR1C3 or a component of a AKR1C3 ternary complex with a protein of interest (POI) and an E3-ligase.

Provided herein are methods of degrading AKR1C3 in a cell, comprising contacting the cell with a compound or a composition as disclosed herein (e.g., the compounds of compounds of Formula 0, Formula I, Formula IA, and Formula II, or pharmaceutically acceptable salts of the compounds) in an amount sufficient to degrade the AKR1C3. The contacting of the cell can occur in vitro or in vivo. In some cases, contacting of the cell occurs in vitro. In other cases, contacting of the cell occurs in vivo. Therefore, the disclosure includes administering one or more of a compound described herein to a subject, such as a human, in need thereof. In some embodiments, the subject suffers from a disease or disorder associated with aberrant activity of a AKR1C3. Disorders associated with aberrant activity of a AKR1C3 include, but are not limited to, cancer (e.g., prostate cancer), inflammatory diseases, and neurological diseases. Specifically contemplated cancers include leukemia, lymphoma, multiple myeloma, breast cancer, prostate cancer, pancreatic cancer, colon cancer, thyroid cancer, bladder cancer, liver cancer, neuroblastoma, brain cancer, lung cancer, ovarian cancer, stomach cancer, skin cancer, cervical cancer, testicular cancer, kidney cancer, carcinoid tumors, or bone cancer.

The disclosed methods utilize compounds that degrade AKR1C3, for treating, e.g., cancer. Methods for assessing the usefulness of a compound for treating cancer are known to those of skill in the art. For example, compounds may be assessed using models of cancer, including cells (such as prostate cancer cells), animal models (such as mouse xenograph or other cancer models), or in human subjects having, e.g., prostate cancer.

In some embodiments, the disclosure provides a method of inhibiting AKR1C3 comprising contacting AKR1C3 with a compound or pharmaceutically acceptable salt of formula (A) disclosed herein in an amount effective to inhibit AKR1C3. Methods of the disclosure can include inhibiting AKR1C3 activity in a subject in need thereof by administration of a compound of formula (A) or a pharmaceutically acceptable salt thereof or a pharmaceutical composition including the same.

The disclosure provides a method of treating, inhibiting, and/or preventing a disease in a subject in need thereof, comprising administering to the subject a therapeutically effective amount of a compound of formula (0) or pharmaceutically acceptable salt thereof. In some cases, the methods comprise administering to the subject a therapeutically effective amount of a compound of formula (I) or pharmaceutically acceptable salt thereof. In some cases, the methods comprise administering to the subject a therapeutically effective amount of a compound of formula (IA) or pharmaceutically acceptable salt thereof. In some cases, the methods comprise administering to the subject a therapeutically effective amount of a compound of formula (II) or pharmaceutically acceptable salt thereof.

The disease can be mediated by AKR1C3 activity. The disease can be cancer. For example, the cancer can be selected from leukemia (e.g., acute lymphoblastic leukemia, acute myeloid leukemia, chronic lymphocytic leukemia, chronic myelogenous leukemia, T-cell acute lymphoblastic leukemia), lymphoma (e.g., Hodgkin lymphoma, non-Hodgkin lymphoma), multiple myeloma, breast cancer, prostate cancer, pancreatic cancer, colon cancer, thyroid cancer, bladder cancer, liver cancer, neuroblastoma, brain cancers (e.g., gliomas, meningiomas, pituitary adenomas, etc.), lung cancer, ovarian cancer, stomach cancer, skin cancer (melanoma), cervical cancer, testicular cancer, kidney cancer, carcinoid tumors, bone cancer, and endometrial cancer. In some cases, the cancer is T-cell acute lymphoblastic leukemia. In some cases, the cancer is prostate cancer.

The disclosure further provides a method of enhancing or potentiating the effectiveness of an active agent comprising administering the active agent in combination with an effective amount of a compound disclosed herein (e.g., a compound of formula (0), a compound of formula (I), a compound of formula (IA), and a compound of formula (II)), including pharmaceutical salts thereof. For example, the active agent can be selected from an anti-cancer drug, including but not limited to navitoclax (ABT-737), daunorubicin, cisplatin, doxorubicin, idarubicin, and dexamethasone.

As used herein, treating refers to alleviating, reducing, and/or stopping of progression of a disease and/or symptoms thereof. As used herein, preventing refers to reducing the chance of contracting or the chance of recurrence of a disease

As used herein, the term “therapeutically effective amount” means an amount of a compound or combination of therapeutically active that ameliorates, attenuates or eliminates one or more symptoms of a particular disease or condition (e.g., parasitic disease), or prevents or delays the onset of one of more symptoms of a particular disease or condition.

As used herein, the terms “subject” and “patient” may be used interchangeably and mean animals, such as dogs, cats, cows, horses, and sheep (e.g., non-human animals) and humans. Particular subjects or patients are mammals (e.g., humans). The terms subject and patient include males and females.

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

The disclosed methods comprise administering a compound of Formula (A) using any suitable route of administration. Illustrative suitable routes of administration include, for example, parenterally, subcutaneously, orally, topically, pulmonarily, rectally, vaginally, intravenously, intraperitoneally, intrathecally, intracerbrally, epidurally, intramuscularly, intradermally, or intracarotidly.

The compositions of the present invention may be administered to a patient and may be conveniently formulated for administration with any pharmaceutically acceptable carrier(s).

Uses of the compounds disclosed herein in the preparation of a medicament for treating, inhibiting, and/or preventing parasitic diseases also are provided herein.

The compounds described herein can be used to decrease or prevent cancer in human subjects with e.g., prostate cancer. In a particular example, a compound or mixture is administered orally, such as by mixing with distilled water. In another example, a test compound or mixture is administered intravenously, such as in saline or distilled water. In some examples, treatment with test compound may be a single dose or repeated doses. The test compound may be administered about every 6 hours, about every 12 hours, about every 24 hours (daily), about every 48 hours, about every 72 hours, or about weekly. Treatment with repeated doses may continue for a period of time, for example for about 1 week to 12 months, such as about 1 week to about 6 months, or about 2 weeks to about 3 months, or about 1 to 2 months. Administration of a compound may also continue indefinitely. Doses of test compound are from about 0.1 mg/kg to about 400 mg/kg, such as about 1 mg/kg to about 300 mg/kg, about 2 mg/kg to 200 mg/kg, about 10 mg/kg to about 100 mg/kg, about 20 mg/kg to about 75 mg/kg, or about 25 mg/kg to about 50 mg/kg.

It will be understood that the methods and compositions described herein for treating cancer, comprising administering a compound that degrades AKR1C3, are applicable to methods of treating other diseases related to AKR1C3 activity, such as those described above. The methods for assessing the effectiveness of test compounds for treating such diseases in cells, appropriate animal models, or affected subjects are known to one of skill in the art.

Uses of the compounds disclosed herein in the preparation of a medicament for treating diseases or disorders related to AKR1C3 activity also are provided herein.

The disclosure herein will be understood more readily by reference to the following examples, below.

### EMBODIMENTS

1. A compound, or pharmaceutically acceptable salt thereof, having the structure of Formula (0):

A-L-B (0),
wherein
   A is a AKR1C3 inhibitor and/or binder moiety;
   B is a proteolysis targeting moiety; and
   L is a linker moiety.

2. The compound or salt of embodiment 1, wherein the AKR1C3 inhibitor and/or binder moiety is selected from a small molecule moiety, a peptide moiety, and an antibody moiety or a fragment thereof.

3. The compound or salt of embodiment 1 or 2, wherein the AKR1C3 inhibitor and/or binder moiety is a small molecule moiety adapted to be coupled to L through an amide moiety.

4. The compound or salt of any one of embodiments 1-3, wherein the AKR1C3 inhibitor and/or binder moiety is a moiety of formula (A1):

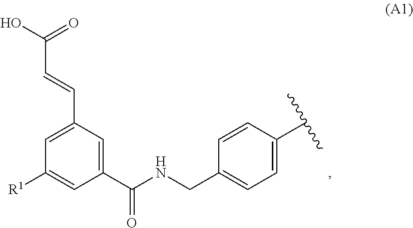

wherein
   R¹ is selected from C₁-C₆alkyl, C₆-C₁₀aryl, C₂-C₆alkenyl, C₁-C₆alkoxy, SO₂R^{s}, (CO)ₘN(R^{a})₂, and 5-10 membered heteroaromatic having 1-3 ring heteroatoms selected from N, O, S;
   R^{s} is C₁-C₆alkyl or substituted or unsubstituted C₆-C₁₀aryl;
   each R^{a} independently is H or C₁-C₆alkyl;
   m is 0 or 1; and
   R¹ is optionally substituted with one or more substituents selected from C₆-C₁₀aryl, 5-15 membered heteroaryl having 1-4 ring heteroatoms selected from N, O, and S, formyl, C₃-C₆cycloalkyl, halo, cycloheteroalkyl-alkylene, hydroxy-C₁-C₃alkylene, methoxymethyl, phenyoxy, cyano, C₁-C₆alkyl, C₁-C₆haloalkyl, and 5-7 membered fused cycloheteroalkyl having 1 to 3 ring heteroatoms selected from N, O, and S.

5. The compound or salt of any one of embodiments 1-4, wherein the AKR1C3 inhibitor and/or binder moiety is selected from

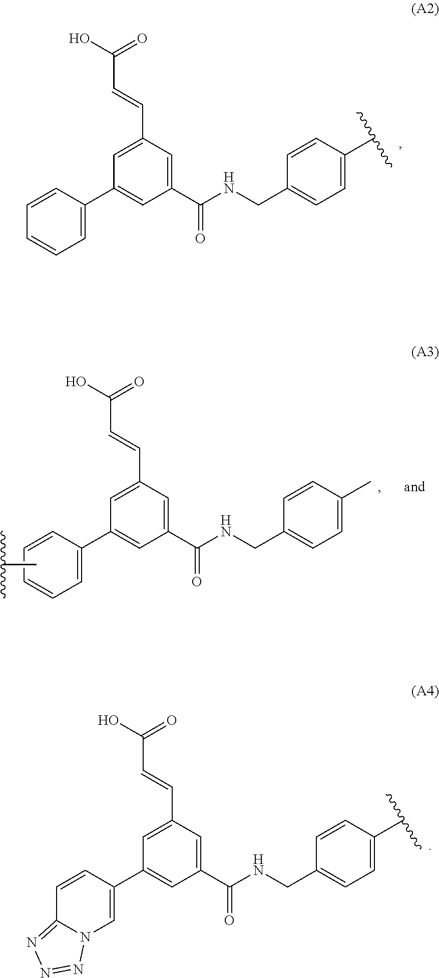

6. The compound or salt of any one of embodiments 1-5, wherein the linker moiety is C1-C₁₈alkylene interrupted with one or more of (i) non-adjacent heteroatom(s) selected from O, S, and NR^{N}, (ii) C₆-C₁₀arylene, (iii) 5-10 membered cycloheteroalkylene having 1-3 ring heteroatoms selected from N, O, and S, and (iv) C(O)NR^{N}, and (v) NR^{N}C(O); and each R^{N} is independently H or C₁-6 alkyl.

7 The compound or salt of any one of embodiments 1-6, wherein L is selected from

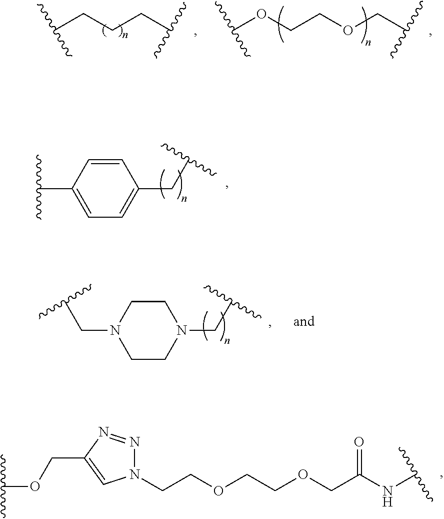

wherein
   n is 0-10.

8. The compound or salt of any one of embodiments 1-7, wherein the proteolysis targeting moiety is selected from a small molecule moiety, a peptide moiety, an electrophilic moiety, and an antibody moiety or a fragment thereof.

9. The compound or salt of any one of embodiments 1-8, wherein the proteolysis targeting moiety is an IκBα-derived moiety, a HIF-1α derived moiety, nutlin, bestatin, methyl-bestatin, thalidomide, pomalidomide, lenalidomide, a thalidomide analog, a VHL binding molecule, an IAP binding molecule, or a terminal alkyne.

10. The compound or salt of any one of embodiments 1-9, wherein the proteolysis targeting moiety comprises a E3 ligase binding moiety capable of binding to and/or recruiting an E3 ligase.

11. The compound or salt of embodiment 10, wherein the E3 ligase binding moiety is VHL, cereblon, MDM2, clAP1, or APC/CCDH-1.

12. The compound or salt of embodiment 11, wherein the E3 ligase binding moiety is cereblon.

13. The compound or salt of any one of embodiments 1-10, wherein the E3 ligase binding moiety has a structure of formula:

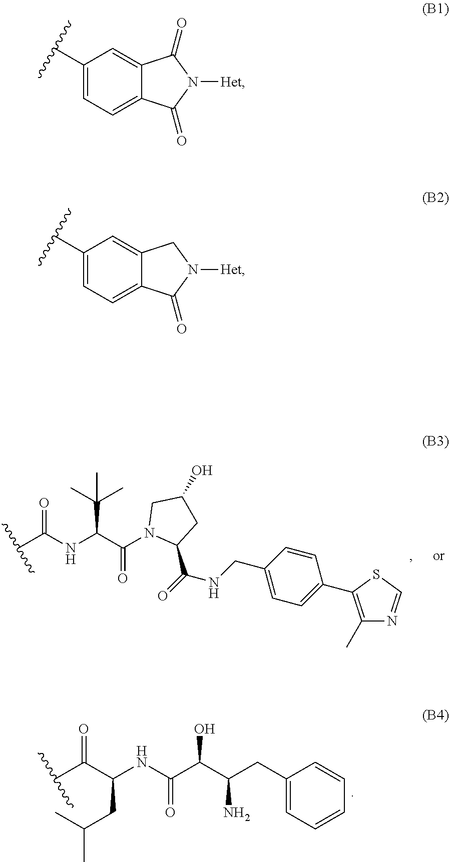

wherein
   Het is a 3-7 membered heterocycloalkyl having 1-3 ring heteroatoms selected from O, S, and N, and said heterocycloalkyl is optionally substituted with one or more oxo.

14. The compound or salt of embodiment 13, wherein the E3 ligase binding moiety has a structure of formula:

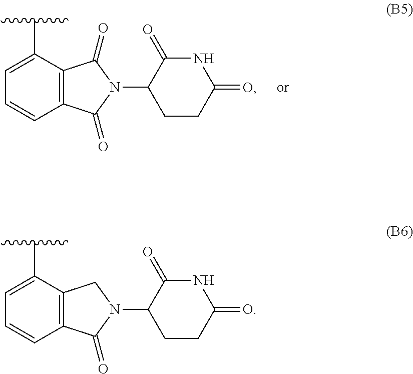 [SMILES: *c1cccc2c1C(=O)N(C1CCC(=O)NC1=O)C2=O ; *c1cccc2c1CN(C1CCC(=O)NC1=O)C2=O]

15. The compound or salt of any one of embodiments 1-9, wherein the proteolysis targeting moiety is a terminal alkyne.

16. The compound or salt of embodiment 4 or 5, having the structure of Formula (I)

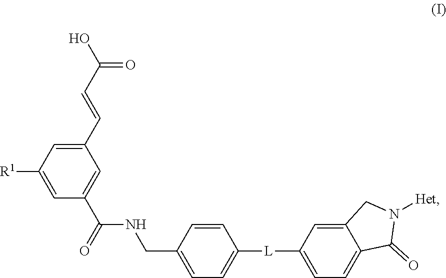

wherein
   L is C₁-C₁₈alkylene interrupted with one or more of (i) non-adjacent heteroatom(s) selected from O, S, and NR^{N}, (ii) C₆-C₁₀arylene, (iii) 5-10 membered cycloheteroalkylene having 1-3 ring heteroatoms selected from N, O, and S, and (iv) C(O)NR^{N}, and (v) NR^{N}C(O);
   each R^{N} is independently H or C₁₋₆ alkyl; and
   Het is a 3-7 membered heterocycloalkyl having 1-3 ring heteroatoms selected from O, S, and N, and said heterocycloalkyl is optionally substituted with one or more oxo.

17. The compound or salt of embodiment 16, wherein R¹ is C₆-C₁₀aryl or 6-10 membered heteroaryl having 1-3 ring heteroatoms selected from N, O, or S.

18. The compound or salt of embodiment 17, wherein R¹ is phenyl or

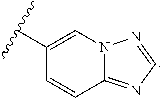 [SMILES: *c1ccc2ncnn2c1]

19. The compound or salt of embodiment 18, wherein R¹ is phenyl.

20. The compound or salt of any one of embodiments 16-19, having the structure of Formula (IA):

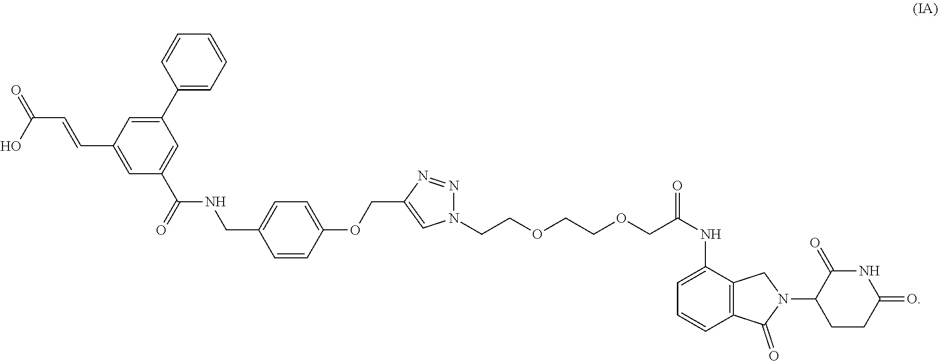 [SMILES: O=C(O)/C=C/c1cc(C(=O)NCc2ccc(OCc3cn(CCOCCOCC(=O)Nc4cccc5c4CN(C4CCC(=O)NC4=O)C5=O)nn3)cc2)cc(-c2ccccc2)c1]

21. The compound or salt of any one of embodiments 1-9, having the structure of Formula (II):

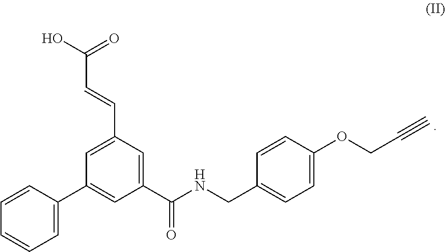 [SMILES: C#CCOc1ccc(CNC(=O)c2cc(/C=C/C(=O)O)cc(-c3ccccc3)c2)cc1]

22. A pharmaceutical composition comprising the compound or salt of any one of embodiments 1-21 and a pharmaceutically acceptable carrier or excipient.

23. A method of inhibiting an aldo-keto reductase family 1 member C3 (AKR1C3) comprising contacting the AKR1C3 with a compound of Formula (0): A-L-B, wherein A is a AKR1C3 inhibitor/binder moiety, B is a proteolysis targeting moiety, and L is a linker moiety.

24. The method of embodiment 23, wherein the proteolysis targeting moiety is selected from a small molecule moiety, a peptide moiety, an electrophilic moiety, and an antibody moiety or a fragment thereof.

25. The method of embodiment 23 or 24, wherein the proteolysis targeting moiety is an IκBα-derived moiety, a HIF-1α derived moiety, nutlin, bestatin, methyl-bestatin, thalidomide, pomalidomide, lenalidomide, a thalidomide analog, a VHL binding molecule, an IAP binding molecule, or a terminal alkyne.

26. The method of any one of embodiments 23-25, wherein the proteolysis targeting moiety is a terminal alkyne.

27. A method of degrading an aldo-keto reductase family 1 member C3 (AKR1C3) comprising administering to a subject in need thereof a therapeutically effective amount of a composition comprising a AKR1C3 inhibitor and/or binder moiety linked to an E3 ligase binding moiety.

28. The method of embodiment 23, wherein the composition comprises the compound or salt of any one of embodiments 1 to 21.

29. A method of treating or preventing a disease or disorder capable of being modulated by AKR1C3 degradation or by androgen receptor splice variant (ARv7), comprising administering to a subject in need thereof a therapeutically effective amount of a composition comprising a AKR1C3 inhibitor moiety linked to an E3 ligase binding moiety.

30. The method of embodiment 29, wherein the composition comprises the compound or salt of any one of embodiments 1 to 21.

31. The method of embodiment 29 or 30, wherein the disease or disorder is selected from cancer, inflammatory diseases, and neurological diseases.

32. The method of embodiment 31, wherein the disease or disorder is cancer, endometriosis, or multiple sclerosis.

33. The method of embodiment 31, wherein the disease or disorder is cancer.

34. The method of embodiment 33, wherein the cancer is leukemia, lymphoma, multiple myeloma, breast cancer, prostate cancer, pancreatic cancer, colon cancer, thyroid cancer, bladder cancer, liver cancer, neuroblastoma, brain cancer, lung cancer, ovarian cancer, stomach cancer, skin cancer, cervical cancer, testicular cancer, kidney cancer, carcinoid tumors, or bone cancer.

35. The method of embodiment 34, wherein the cancer is prostate cancer.

36. The method of embodiment 34, wherein the lymphoma is Hodgkin lymphoma or non-Hodgkin lymphoma.

37. The method of embodiment 34, wherein the brain cancer is a glioma, a meningioma, or a pituitary adenoma.

38. The method of embodiment 34, wherein the skin cancer is melanoma.

39. The method of embodiment 34, wherein the leukemia is acute lymphoblastic leukemia, acute myeloid leukemia, chronic lymphocytic leukemia, T-cell acute lymphoblastic leukemia, or chronic myelogenous leukemia.

40. The method of any one of embodiments 23-39, further comprising administration of an additional therapeutic agent.

41. The method of embodiment 40, wherein the additional therapeutic agent comprises an anti-cancer drug.

42. A composition comprising a AKR1C3 inhibitor and/or binder moiety linked to an E3 ligase binding moiety for use in degrading AKR1C3.

43. A pharmaceutical composition comprising the compound or salt of any one of embodiments 1-21 and a pharmaceutically acceptable carrier or excipient for use in degrading AKR1C3 or ARv7.

44. A composition comprising a AKR1C3 inhibitor and/or binder moiety linked to an E3 ligase binding moiety for use in treating or preventing a disease or disorder capable of being modulated by AKR1C3 degradation or by ARv7 degradation.

45. The composition for use of embodiment 44, wherein the disease or disorder is selected from the group consisting of cancer, inflammatory diseases, and neurological diseases.

46. The composition for use of embodiment 45, wherein the disease or disorder is cancer, endometriosis, or multiple sclerosis.

47. The composition for use of embodiment 46, wherein the disease or disorder is cancer.

48. The composition for use of embodiment 47, wherein the cancer is leukemia, lymphoma, multiple myeloma, breast cancer, prostate cancer, pancreatic cancer, colon cancer, thyroid cancer, bladder cancer, liver cancer, neuroblastoma, brain cancer, lung cancer, ovarian cancer, stomach cancer, skin cancer, cervical cancer, testicular cancer, kidney cancer, carcinoid tumors, or bone cancer.

49. The composition for use of embodiment 48, wherein the cancer is prostate cancer.

50. The composition for use of embodiment 48, wherein the lymphoma is Hodgkin lymphoma or non-Hodgkin lymphoma.

51. The composition for use of embodiment 48, wherein the brain cancer is a glioma, a meningioma, or a pituitary adenoma.

52. The composition for use of embodiment 48, wherein the skin cancer is melanoma.

53. The composition for use of embodiment 48, wherein the leukemia, is acute lymphoblastic leukemia, acute myeloid leukemia, chronic lymphocytic leukemia, T-cell acute lymphoblastic leukemia, or chronic myelogenous leukemia.

The foregoing summary is not intended to define every aspect of the disclosure, and additional aspects are described in other sections of the disclosure. The entire document is intended to be related as a unified disclosure, and it should be understood that all combinations of features described herein are contemplated, even if the combination of features are not found together in the same sentence, or paragraph, or section of this document.

In addition to the foregoing, the disclosure includes, as an additional aspect, all embodiments of the disclosure narrower in scope in any way than the variations specifically mentioned above. With respect to aspects of the disclosure described or claimed with “a” or “an,” these terms mean “one or more” unless context unambiguously requires a more restricted meaning. With respect to elements described as one or more within a set, all combinations within the set are contemplated as combination inventions. If aspects of the disclosure are described as “comprising” a feature, embodiments also are contemplated “consisting of” or “consisting essentially of” the feature.

Aspects of the disclosure described as methods of treatment should also be understood to include first or subsequent “medical use” aspects of the disclosure or “Swiss use” of compositions for the manufacture of a medicament for treatment of the same disease or condition.

Multiple embodiments are contemplated for combinations described herein. For example, some aspects of the disclosure that are described as a method of treatment (or medical use) combining two or more compounds or agents, whether administered separately (sequentially or simultaneously) or in combination (co-formulated or mixed). For each aspect described in this manner, the disclosure further includes a composition comprising the two or more compounds or agents co-formulated or in admixture with each other; and the disclosure further includes a kit or unit dose containing the two or more compounds/agents packaged together, but not in admixture. Optionally, such compositions, kits or doses further include one or more carriers in admixture with one or both agents or co-packaged for formulation prior to administration to a subject. The reverse also is true: some aspects of the disclosure are described herein as compositions useful for therapy and containing two or more therapeutic agents. Equivalent methods and uses are specifically contemplated.

Although the applicant(s) invented the full scope of the claims appended hereto, the claims appended hereto are not intended to encompass within their scope the prior art. Therefore, in the event that statutory or judicially recognized prior art within the scope of a claim is brought to the attention of the applicants by a Patent Office or other entity or individual, the applicant(s) reserve the right to exercise amendment rights under applicable patent laws to redefine the subject matter of such a claim to specifically exclude such prior art or obvious variations of statutory prior art from the scope of such a claim. Variations of the disclosure defined by such amended claims also are intended as aspects of the invention. Additional features and variations of the invention will be apparent to those skilled in the art from the entirety of this application, and all such features are intended as aspects of the disclosure.

The disclosure herein will be understood more readily by reference to the following examples, below.

### EXAMPLES

The following examples are provided for illustration and are not intended to limit the scope of the disclosure.

### General Experimental Procedures:

General Chemistry Procedures. All reactions were carried out in oven-dried glassware under positive nitrogen pressure unless otherwise noted. Reaction progress was monitored by thin-layer chromatography carried out on silica gel plates (2.5 cm×7.5 cm, 200 μm thick, 60 F254) and visualized by using UV (254 nm) or by dragendorff solution as indicator. Flash column chromatography was performed with silica gel (40-63 μm, 60 Å) using the mobile phase indicated. Commercial grade solvents and reagents were purchased from Fisher Scientific (Houston, TX) or Sigma-Aldrich (Milwaukee, WI) and were used without further purification. Anhydrous solvents were purchased from Across Organics and stored under an atmosphere of dry nitrogen over molecular sieves.

¹H and ¹³C NMR spectra were recorded in the indicated solvent on a Bruker 400 MHz Advance III HD spectrometer at 400 and 100 MHz for ¹H and ¹³C, respectively. Multiplicities are indicated by s (single), d (doublet), t (triplet), m (multiplet), and br (broad). Chemical shifts (o) are reported in parts per million (ppm) and coupling constants (J), in hertz.

High-resolution mass spectra (HRMS) were recorded with an Agilent 6230 LC/TOF spectrometer using an ESI source coupled to an Agilent Infinity 1260 system running in reverse phase with a ZORBAX RRHT Extend-C18 (80 Å, 2.1×50 mm, 1.8 μm) column using solvent A (water with 0.1% Formic acid), solvent B (acetonitrile with 0.1% Formic acid), and a flow rate of 0.6 mL/min starting a mixture of 95% A and 5% B. Solvent B is gradually increased to 95% at 5 min, held at 95% until 6 min, then gradually ramped back down to 5% at 8.0 min. The purity analysis of final compounds were determined ≥95% pure using a Waters ACQUITY ultra-performance liquid chromatography (UPLC) H-Class System with TUV (254 nm) detector and Empower 2 software (Milford, MA, USA) using an Agilent Eclipse plus C18 5μ column (4.6×150 mm). Chromatography was performed using solvent A (water with 0.1% Trifluoroacetic acid), solvent B (methanol with 0.1% Trifluoroacetic acid), and a flow rate of 1.0 mL/min for 20 min. with an isocratic system (20:80, A:B)

Enzyme Inhibition Assay. (S)-(+)-1,2,3,4-tetrahydro-1-naphthol (S-tetralol) was purchased from Sigma-Aldrich (St. Louis, MO). Nicotinamide adenine dinucleotide (NAD⁺) and nicotinamide adenine dinucleotide phosphate (NADP⁺) were purchased from Roche Diagnostics (Indianapolis, IN). Homogeneous recombinant enzymes AKR1C1, AKR1C2, AKR1C3 and AKR1C4 were prepared and purified as previously described.³ The specific activities of AKR1C3 and AKR1C2 for the oxidation of S-tetralol are 2.0 and 1.5 μmol/(min mg), respectively.

Assay of Enzyme Activity. The dehydrogenase activities of AKR1C3 and AKR1C2 were determined by measuring the UV absorption of NADH formation at 340 nm using a Beckman DU-640 spectrophotometer. A typical assay solution contained 100 mM potassium phosphate pH 7.0, 2.3 mM NAD⁺, 3.0 mM (S)-(+)-1,2,3,4-tetrahydro-1-naphthol (S-tetralol), and 4% acetonitrile (v/v). The mixtures were incubated at 37 °C for 3 min followed by adding a serial dilution of AKR1C1, AKR1C2, AKR1C3 or AKR1C4 solution to a final volume of 1 mL to initiate the reaction. After continuously monitoring for 5 min, we recorded the increase in UV absorption using different concentrations of an enzyme to calculate the initial velocity and determine the specific activity of the enzyme.

IC₅₀ Value Determination. The inhibitory potency for each compound was represented by IC50 values and measured as described before. The IC50 values of baccharin and baccharin analogues were determined by measuring their inhibition of the NADP+-dependent oxidation of S-tetralol catalyzed by AKR1C3 or AKR1C2. The concentrations of S-tetralol used in this assay for AKR1C3 and AKR1C2 were 165 μM and 15 μM, respectively, which were equal to the Km value for each enzyme isoform to make a direct comparison of IC50 values. The IC50 value of each compound was acquired from a single experiment with each inhibitor concentration run in quadruplicate and directly calculated by fitting the inhibition data to an equation [y=(range)/[1+ (I/IC50)S]+background] using Grafit 5.0 software. In this equation, “range” is the fitted uninhibited value minus the “background” and “S” is a slope factor. “I” is the concentration of the inhibitor. The equation assumes that y decreases with the increasing “I”.

Cell culture and reagents. 22Rv1 cells were purchased from the American Type Culture Collection (ATCC in 2016) and cultured in RPMI 1640 supplemented with 10% Fetal Bovine Serum (FBS, Fisher Scientific, MT35011CV) and Penicillin-Streptomycin Solution (1%, Fisher Scientific, MT30001Cl) at 37 °C in a humidified incubator with 5% carbon dioxide atmosphere. Stock solutions of E3 ligase ligand, lenalidomide 6 (10 mM), PROTAC 5 (25 mM), and AKR1C3 inhibitor 3 (100 mM) were prepared in DMSO.

Cell viability assay. Cells were seeded at a density of 10,000 cells/well in a 96-well plate containing 100 μL growth media per well and were incubated at 37 °C in a humidified incubator with 5% carbon dioxide over ~18-24 hours. Cells were treated with 6, 5, and 3 serially diluted at the indicated concentrations limiting the final DMSO concentration to less than 0.1% and incubated at 37 °C for 72 hours. 10 μL of MTS reagent (CellTiter96®Aqueous One Solution Reagent, Promega, G3580) was added to each well and incubated at the above-mentioned conditions for 4 hours. Absorbance was recorded at OD 490 nm on a Synergy LX multi-mode reader and the viability of cells were plotted as percentage of DMSO control.

Protein Expression via Western Blot. 22Rv1 cells were seeded in a 6-well plate and incubated overnight. Next day, the cells were treated with varying concentrations of 6 (1 and 10 μM) and 5 (0.5, 1, 10, 50, 100, 250, and 500 nM; 1, 10, and 50 μM) for 24 hours and 5 (1 and 10 nM) at different time points (0, 2, 4, 6, 12, 16, 24, 48, and 72 h). Whole cell lysates were extracted in RIPA lysis buffer containing protease inhibitor and EDTA (Fisher Scientific, PI78440). Protein concentrations in each sample was estimated following BCA assay (Pierce™ BCA protein assay kit, Fisher Scientific, PI23227) as per the manufacturer's protocol. Proteins were standardized using RIPA lysis buffer with Laemelli SDS sample buffer (Thermo Scientific, AAJ61337AC) and heated at 1000C for 15 minutes. The proteins were resolved on a 4-12% premade gel (Invitrogen, NW04120BOX) at 60-90 V in 20× Bolt™ MES SDS running buffer (Fisher Scientific, B000202) and transferred onto a nitrocellulose membrane (Invitrogen, IB23001) using iBlot 2. The membranes were blocked with 2.5% non-fat dry milk (Bio-Rad, 1706404) for 1 h, and the membrane was incubated overnight on a rocking platform at 4 °C with the desired primary antibodies against AKR1C3 (mouse mAb, 1:200, Sigma, A6229), AKR1C1/C2 (rabbit mAb, 1:500, Abcam, ab179448), ARv7 (rabbit mAb, 1:500, Abcam, ab198394) and actin (mouse mAb, 1:1,000, ThermoFisher, MA5-11869). The membranes were washed three times, 10 minutes each, with 1×Tris-buffered saline (TBST, Bio-Rad, 1706435) in 0.1% Tween 20, on a rocking platform wherein they were incubated with rabbit anti-mouse IgG (ThermoFisher, 31450) or goat anti-rabbit IgG (Jackson ImmunoResearch, 111-035-003) secondary antibody (1:1,000) horseradish peroxidase conjugate for 1 hour. The membranes were again washed three times with 1×TBST, upon which they were exposed to LI-COR WesternSure PREMIUM chemiluminescent substrate (Fisher Scientific, 50-489-552) and developed in a dark room with Konica Minolta equipment. Bands were quantified by densitometry using ImageJ software and fold change in AKR1C3, AKR1C1/C2, and ARv7 protein expression was determined based on actin controls.

Docking Studies. Docking experiments were performed with SeeSAR 12.1 software (BioSolveIT, Sankt Augustin, Germany). The crystal structure of AKR1C3 bound with the reference ligand indomethacin (PDB ID: 3UG8) was imported into the binding site tool as a PDB file. The reference ligand was removed, and the binding site defined as a 30 amino acid residue pocket directly surrounding the template ligand. The default parameter settings of SeeSAR were employed. Compounds were prepared and docked with FlexX, wherein fragments are placed into multiple places in the defined pocket and scored with a pre-scoring system.5 FlexS,6 was used to generate compound/reference ligand superimpositioning to determine similarity between the test compound and the reference ligand, providing a ranked list for prioritizing compounds. Binding poses were scored by hydrogen dehydration (HYDE) 7 and the top 20 scoring binding poses of each compound were imported and analyzed in SeeSAR. The top scoring pose was selected based on estimated affinity, ligand efficiency, and torsion energy. Docking figure is generated from a perspective to illustrate binding interactions in the most accessible way in a 2D figure.

### Synthesis of Compounds

Scheme 1 depicts an illustrative synthesis of compounds 1-8, which are useful for preparing a compound of Formula (II) disclosed herein.

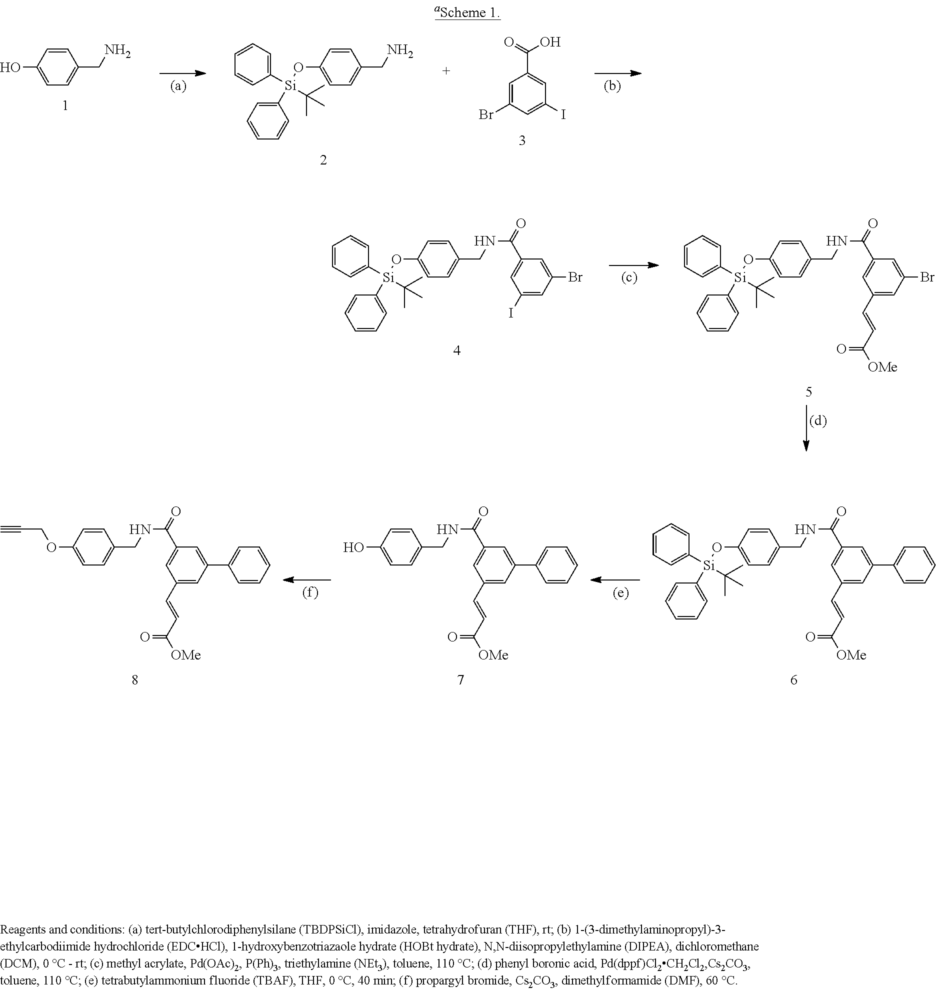

(4-((tert-butyldiphenylsilyl)oxy)phenyl)methanamine (2): To a stirred solution of 4-(aminomethyl) phenol (1) (1.85 g, 15.0 mmol, 1 equiv) in anhydrous toluene (75 mL) imidazole (2.04 g, 30.0 mmol, 2 equiv) was added followed by the addition of tert-butylchlorodiphenylsilane (TBDPSiCl) (5.8 mL, 22.5 mmol, 1.5 equiv). The mixture was stirred overnight at room temperature under a N₂ atmosphere. Water was added and the solution was extracted with DCM. The organic layer was collected, dried over anhydrous Na₂SO₄, filtered, and concentrated in vacuo. The crude product was purified by flash column chromatography using hexane/EtOAc as the eluents to afford the titled compound as a yellow solid (3.09 g, 57%). R_{f}=0.07 (hexane/EtOAc=9:1). ¹H NMR (400 MHz, CDCl₃): δ_{H} 1.15 (9H, s), 1.86 (2H, s), 3.75 (2H, s), 6.77 (d, J=8.0 Hz, 2H), 7.05 (d, J=8.0 Hz, 2H) 7.38-7.46 (6H, m), 7.76 (d, J=8.0 Hz, 4H). ¹³C NMR (100 MHz, CDCl₃): δ_{c} 19.5, 26.6, 45.8, 119.7, 127.8, 128.1, 129.9, 133.0, 135.5, 135.5, 154.5.

3-bromo-N-(4-((tert-butyldiphenylsilyl)oxy)benzyl)-5-iodobenzamide (4): To a stirred solution of 3-bromo-5-iodobenzoic acid (3) (0.72 g, 2.2 mmol, 1 equiv) in anhydrous DCM (12 mL) was added EDC·HCl (0.51 g, 2.7 mmol, 1.2 equiv) and HOBt hydrate (0.44 g, 2.7 mmol, 1.2 equiv) at 0 °C under a N₂ atmosphere. At room temperature DIPEA (0.86 g, 6.6 mmol, 3 equiv) and 2 (0.80 g, 2.2 mmol, 1 equiv) was added, the mixture stirred overnight at room temperature under a N₂ atmosphere. The reaction mixture was washed with a saturated solution of NH₄Cl, water, and extracted with DCM. The organic layer was collected, dried over anhydrous Na₂SO₄, filtered, and concentrated in vacuo to afford the titled compound as a tan solid (1.29 g, 87%). R_{f}=0.43 (hexane/EtOAc=6:1). ¹H NMR (400 MHz, CDCl₃): δ_{H} 1.13 (9H, s), 4.46 (d, J=4.0 Hz, 2H), 6.41 (t, J=6.0 Hz, 1H), 6.75 (d, J=12.0 Hz, 2H), 7.05 (d, J=12.0 Hz, 2H), 7.37-7.47 (7H, m), 7.73 (d, J=8.0 Hz, 4H), 7.85 (1H, s), 7.96 (d, J=16.0 Hz, 2H). ¹³C NMR (100 MHz, CDCl₃): δ_{c} 19.5, 26.5, 43.2, 43.9, 94.5, 120.0, 120.4, 123.2, 127.8, 128.5, 129.1, 129.6, 129.9, 130.0, 130.1, 132.6, 132.8, 134.7, 135.5, 137.8, 142.4, 155.3, 164.3.

Methyl (E)-3-(3-bromo-5-((4-((tert-butyldiphenylsilyl)oxy)benzyl) carbamoyl)phenyl)acrylate (5): To a stirred solution of (4) (2.1 g, 3.1 mmol, 1 equiv) in anhydrous toluene (42 mL) was added methyl acrylate (0.42 mL, 4.7 mmol, 1.5 equiv), P(Ph)₃ (0.08 g, 0.3 mmol, 0.1 equiv), NEt₃ (1.31 mL, 9.4 mmol, 3 equiv) and Pd(OAc)₂ (0.07 g, 0.3 mmol, 0.1 equiv) and the reaction refluxed overnight under a N₂ atmosphere. While the reaction mixture was still hot, the contents were filtered. The filtrate was then allowed to cool and was filtered through a Celite® pad with DCM. The reaction mixture was washed with a saturated solution of NH₄Cl, water, and extracted with DCM. The organic layer was collected, dried over anhydrous Na₂SO₄, filtered, and concentrated in vacuo. The crude product was purified by flash column chromatography using DCM/MeOH as the eluents to afford the titled compound as a brown semi-solid (0.60 g, 30%). R_{f}=0.1 (DCM/MeOH=1:0). ¹H NMR (400 MHz, CDCl₃): δ_{H} 1.10 (9H, s), 3.80 (3H, s), 4.47 (d, J=4.0 Hz, 2H), 6.36 (1H, br. s), 6.43 (d, J=16.0 Hz, 1H), 6.73 (d, J=8.0 Hz, 2H), 7.05 (d, J=8.0 Hz, 2H), 7.35-7.44 (6H, m), 7.56 (d, J=16.0 Hz, 1H), 7.70 (d, J=8.0 Hz, 5H), 7.80 (d, J=12.0 Hz, 1H). ¹³C NMR (100 MHz, CDCl₃): δ_{c} 19.5, 26.5, 43.9, 52.0, 120.0, 120.5, 123.2, 125.3, 127.8, 129.1, 129.9, 130.0, 131.3, 132.7, 133.3, 135.5, 136.8, 137.0, 142.1, 155.3, 165.2, 166.7.

Methyl (E)-3-(5-((4-((tert-butyldiphenylsilyl)oxy)benzyl) carbamoyl)-[1,1 [symbol] biphenyl]-3-yl)acrylate (6): To a stirred solution of (5) (0.8 g, 1.3 mmol, 1 equiv) in anhydrous toluene (45 mL) was added phenyl boronic acid (0.24 g, 2.0 mmol, 1.5 equiv), Pd(dppf)Cl₂·CH₂Cl₂ (0.11 g, 0.13 mmol, 0.1 equiv), and Cs₂CO₃ (0.85 g, 2.6 mmol, 2 equiv) and the reaction refluxed overnight under a N₂ atmosphere. While the reaction mixture was still hot, the contents were filtered, and the filtrate was concentrated in vacuo. The concentrated filtrate was washed with hexane (5×), dissolved in diethyl ether, concentrated in vacuo, washed again with hexane (2×), and concentrated in vacuo. The crude product was purified by flash column chromatography using DCM/MeOH as the eluents to afford the titled compound as a white solid (0.16 g, 18%). R_{f}=0.30 (DCM/MeOH=1:0). ¹H NMR (400 MHz, CDCl₃): δ_{H} 1.12 (9H, s), 3.84 (3H, s), 4.54 (d, J=8.0 Hz, 2H), 6.40 (t, J=6.0 Hz, 1H), 6.54 (d, J=16.0 Hz, 1H), 6.76 (d, J=8.0 Hz, 2H), 7.10 (d, J=8.0 Hz, 2H), 7.36-7.50 (9H, m), 7.59 (d, J=8.0 Hz, 2H), 7.73 (d, J=4.0 Hz, 5H), 7.78 (d, J=20.0 Hz, 2H), 7.88 (1H, s), 7.96 (1H, s). ¹³C NMR (100 MHz, CDCl₃): δ_{c} 19.5, 26.5, 43.8, 51.9, 119.4, 120.0, 125.1, 127.2, 127.3, 127.8, 128.2, 129.0, 129.1, 129.5, 130.0, 130.2, 132.8, 135.4, 135.5, 135.9, 139.5, 142.5, 143.7, 155.3, 166.6, 167.1.

Methyl (E)-3-(5-((4-hydroxybenzyl) carbamoyl)-[1,1 [symbol] biphenyl]-3-yl)acrylate (7): To a stirred solution of (6) (0.23 g, 0.37 mmol, 1 equiv) in anhydrous THF (5 mL) was added TBAF (1 M solution in THF, 0.16 mL, 0.55 mmol, 1.50 equiv) at 0 °C. The reaction was stirred for 40 minutes at 0 °C. Water was added and the solution was extracted with EtOAc and washed with brine. The organic layer was collected, dried over anhydrous Na₂SO₄, filtered, and concentrated in vacuo. The residue was washed with hexane/EtOAc (50:1, 40:1) resulting in solid precipitation. The solid was left in hexane (15 mL) overnight. The solid was then washed with hexane/EtOAc (2×1:0, 3×1:1). The overall yield of the titled compound as a white solid was 60% (0.1 g). R_{f}=0.46 (hexane/EtOAc=1:1). ¹H NMR (400 MHz, (CD₃)₂SO): δ_{H} 3.75 (3H, s), 4.43 (d, J=4.0 Hz, 2H), 6.73 (d, J=8.0 Hz, 2H), 6.83 (d, J=16.0 Hz, 1H), 7.17 (d, J=8.0 Hz, 2H), 7.41 (t, J=6.0 Hz, 1H), 7.49 (t, J=8.0 Hz, 2H), 7.78 (t, J=8.0 Hz, 3H), 8.17 (1H, s), 8.21 (2H, s), 9.10 (t, J=6.0 Hz, 1H), 9.34 (1H, s). ¹³C NMR (100 MHz, (CD₃)₂SO): δ_{c} 42.8, 52.1, 115.5, 119.7, 126.0, 127.4, 127.7, 128.6, 129.2, 129.5, 129.8, 130.0, 135.4, 136.1, 139.3, 141.4, 144.3, 156.8, 165.8, 167.1.

Methyl (E)-3-(5-((4-(prop-2-yn-1-yloxy)benzyl) carbamoyl)-[1,1 [symbol] biphenyl]-3-yl)acrylate (8): To a stirred solution of 7 (0.23 g, 0.59 mmol, 1 equiv), in anhydrous DMF was added Cs₂CO₃ (0.25 g, 0.78 mmol, 1.31 equiv) and the mixture refluxed under a N₂ atmosphere. After 10 min, propargyl bromide (0.04 mL, 0.41 mmol, 0.69 equiv) was added. The reaction mixture refluxed for 6 h. The reaction mixture was filtered, water was added, and the reaction mixture was extracted with EtOAc. The organic layer was collected, dried over anhydrous Na₂SO₄, filtered, and concentrated in vacuo. The residue was then extracted with diethyl ether and washed with water. The organic layer was collected, dried over anhydrous Na₂SO₄, filtered, and concentrated in vacuo and left to dry at room temperature overnight. The residual DMF was removed by washing with copious amounts of water in toluene, dried over anhydrous Na₂SO₄, filtered, and concentrated in vacuo to afford the titled compound as a white solid (0.19 g, 75%). R_{f}=0.75 (hexane/EtOAc=1:1) ¹H NMR (400 MHz, CDCl₃): δ_{H} 2.53 (t, J=2.4 Hz, 1H), 3.82 (3H, s), 4.61 (d, J=8.0 Hz, 2H), 4.69 (d, J=4.0 Hz, 2H), 6.53 (d, J=16.0 Hz, 1H), 6.65 (t, J=4.0 Hz, 1H), 6.97 (d, J=8.0 Hz, 2H), 7.32 (d, J=8.0 Hz, 2H), 7.39-7.43 (m, 1H), 7.45-7.49 (m, 2H), 7.59 (d, J=4.0 Hz, 2H), 7.73 (d, J=16.0 Hz, 1H), 7.82 (t, J=1.4 Hz, 1H), 7.90 (t, J=1.4 Hz, 1H), 7.99 (t, J=1.6 Hz, 1H). ¹³C NMR (100 MHz, CDCl₃): δ_{c} 43.8, 51.9, 55.9, 75.6, 78.5, 115.2, 119.4, 125.1, 127.2, 127.4, 128.2, 129.0, 129.4, 129.6, 131.0, 135.4, 135.8, 139.5, 142.5, 143.7, 157.1, 166.7, 167.1.

(E)-3-(5-((4-Methylbenzyl) carbamoyl)-[1,1 [symbol] biphenyl]-3-yl)acrylic acid. The titled compound was afforded as a white solid (68 mg, 71%). ¹H NMR (400 MHz, (CD₃)₂SO): δ_{H} 2.29 (3H, s), 4.49 (d, J=5.6 Hz, 2H), 6.74 (d, J=16.0 Hz, 1H), 7.15 (d, J=8.0 Hz, 2H), 7.25 (d, J=8.0 Hz, 2H), 7.43 (t, J=7.2 Hz, 1H), 7.52 (t, J=7.6 Hz, 2H), 7.72 (d, J=16.0 Hz, 1H), 7.81 (d, J=7.2 Hz, 2H), 8.15 (1H, s), 8.20 (2H, s), 9.18 (t, J=5.6 Hz, 1H). ¹³C NMR (100 MHz, (CD₃)₂SO): δ_{c} 21.14, 42.99, 121.45, 125.79, 127.46, 127.81, 128.53, 129.34, 129.46, 129.76, 135.74, 135.95, 136.35, 136.88, 137.03, 139.40, 141.43, 143.45, 165.90, 168.01. ESI-HRMS (m/z): [M+H]⁺ calcd for C₂₄H₂₂NO₃, 372.1594; found, 372.1595.

Scheme 2 depicts an illustrative synthesis of compound 12, which is suitable for preparing compounds of the disclosure.

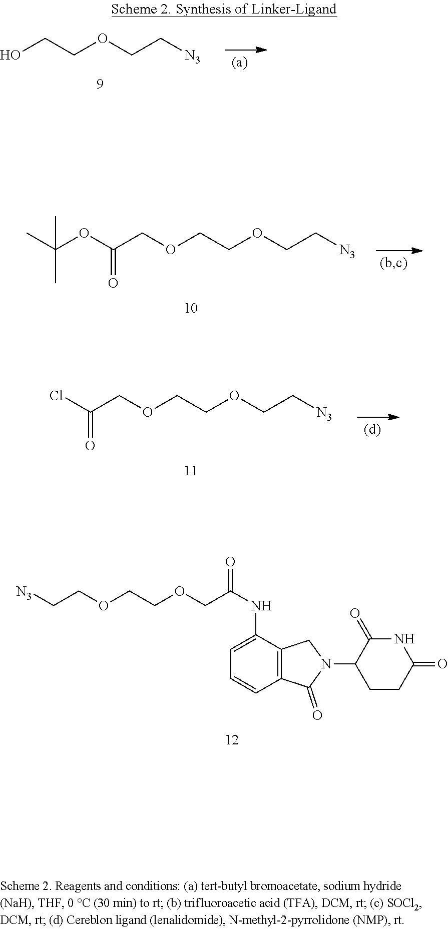 [SMILES: [N-]=[N+]=NCCOCCO ; CC(C)(C)OC(=O)COCCOCCN=[N+]=[N-] ; [N-]=[N+]=NCCOCCOCC(=O)Cl ; [N-]=[N+]=NCCOCCOCC(=O)Nc1cccc2c1CN(C1CCC(=O)NC1=O)C2=O]

tert-butyl 2-(2-(2-azidoethoxy)ethoxy)acetate (10): Under a N₂ atmosphere, to a stirred solution of NaH (60% dispersion in mineral oil, 0.59 g, 14.87 mmol, 1.51 equiv) in anhydrous THF (35 mL) was added a stirred solution of 2-(2-azidoethoxy) ethanol (9) (1.29 g, 9.85 mmol, 1 equiv) in anhydrous THF (69 mL) at 0 °C. After the reaction mixture stirred for 30 min at 0 °C, tert-butyl bromoacetate (2.88 mL, 19.69 mmol, 2 equiv) was added. The mixture stirred for 24 hr at room temperature under a N₂ atmosphere. The reaction mixture was quenched with water (350 mL) and when the effervescence ceased the solution was extracted with EtOAc. The organic layer was collected, dried over anhydrous Na₂SO₄, filtered, and concentrated in vacuo. The crude product was purified by flash column chromatography using Toluene/EtOAc as the eluents to afford the titled compound as a yellow oil (0.72 g, 30%). R_{f}=0.29 (Toluene/EtOAc=1:0). ¹H NMR (400 MHz, CDCl₃): δ_{H} 1.48 (9H, s), 3.39 (t, J=6.0 Hz, 2H), 3.68-3.75 (6H, m), 4.04 (2H, s). ¹³C NMR (100 MHz, CDCl₃): δ_{c} 28.1, 50.7, 69.1, 70.0, 70.7, 70.8, 81.6, 169.6.

To a stirred solution of 10 (0.26 g, 1.06 mmol, 1 equiv) in DCM (0.51 mL) was added TFA (0.25 mL) and the reaction stirred at room temperature for 2 h under a N₂ atmosphere. The reaction mixture was then concentrated in vacuo. At 0 °C, the reaction mixture was diluted with DCM (1.30 mL) followed by dropwise addition of SOCl₂. The reaction stirred at room temperature for 2 h under a N₂ atmosphere. The solvent was concentrated in vacuo to afford 11 (dark brown oil) which was used in the following steps without purification.

2-(2-(2-azidoethoxy)ethoxy)-N-(2-(2,6-dioxopiperidin-3-yl)-1-oxoisoindolin-4-yl) acetamide (12): To solution 11 was added NMP (2.57 mL) and the cereblon ligand (lenalidomide) (0.16 g, 0.63 mmol, 0.5 equiv). The reaction mixture stirred at room temperature overnight under a N₂ atmosphere. Water was added and the solution was extracted with EtOAc and washed with brine. The organic layer was collected, dried over anhydrous Na₂SO₄, filtered, and concentrated in vacuo. The crude product was purified by flash column chromatography using DCM/MeOH as the eluents to afford the titled compound as an off white solid (0.18 g, 37%). R_{f}=0.47 (DCM/MeOH=14:1). ¹H NMR (400 MHz, CDCl₃): δ_{H} 2.18-2.22 (1H, m), 2.35-2.40 (1H, m), 2.81-2.86 (2H, m), 3.38 (t, J=4.0 Hz, 2H), 3.71-3.76 (4H, m), 3.81-3.83 (2H, m), 4.19 (2H, s), 4.46 (2H, s), 5.20-5.25 (m, 1H), 7.49 (t, J=16.0 Hz, 1H), 7.67 (d, J=8.0 Hz, 1H), 7.76 (d, J=8.0 Hz, 1H), 8.52 (1H, s), 8.71 (1H, s). ¹³C NMR (100 MHz, CDCl₃): δ_{c} 23.3, 31.5, 46.5, 50.5, 51.9, 70.1, 70.1, 70.5, 71.1, 121.6, 126.3, 129.2, 131.8, 132.9, 134.7, 168.1, 168.9, 169.7, 171.4.

Scheme 3 depicts an illustrative synthesis of Compound IA.

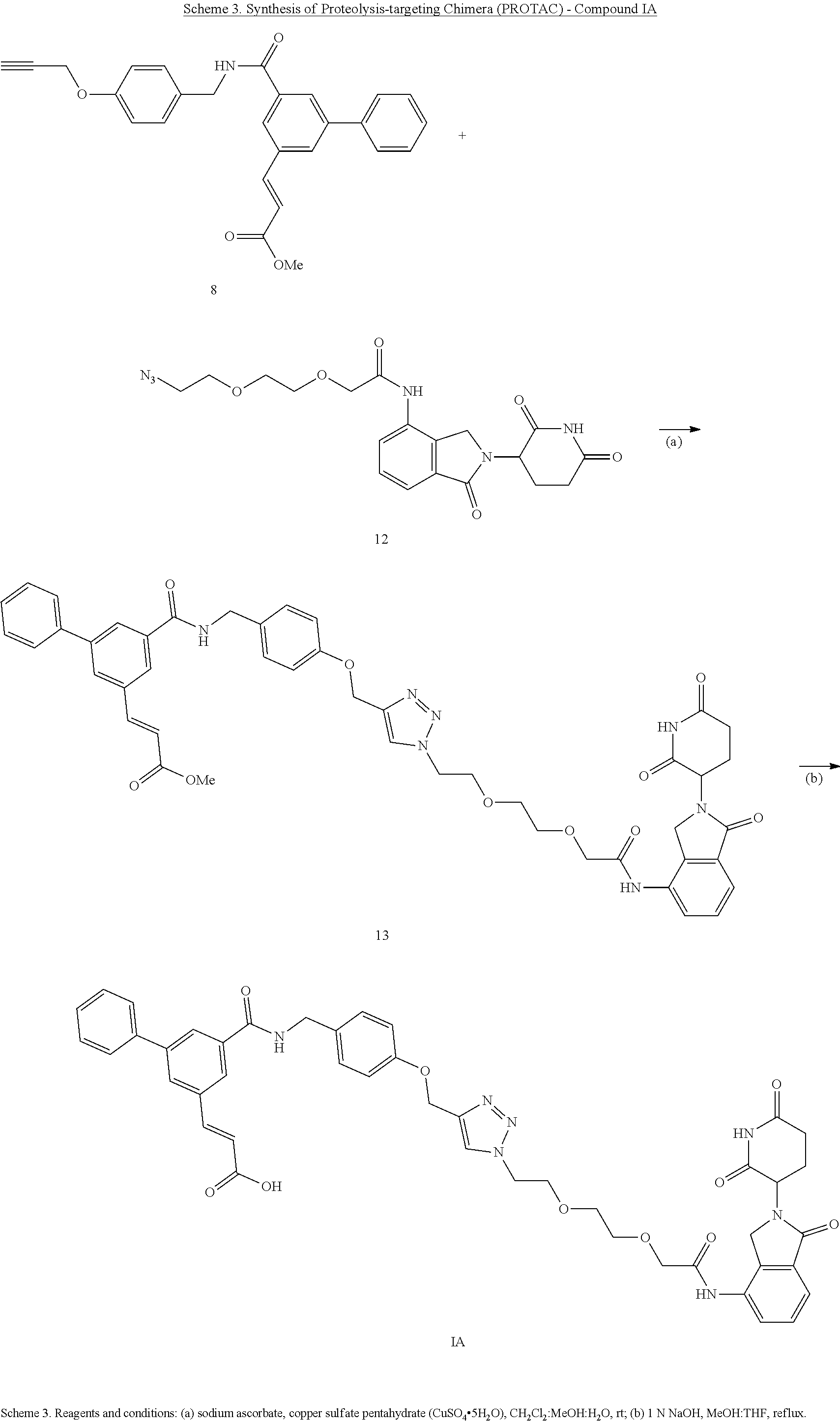 [SMILES: C#CCOc1ccc(CNC(=O)c2cc(/C=C/C(=O)OC)cc(-c3ccccc3)c2)cc1 ; [N-]=[N+]=NCCOCCOCC(=O)Nc1cccc2c1CN(C1CCC(=O)NC1=O)C2=O ; COC(=O)/C=C/c1cc(C(=O)NCc2ccc(OCc3cn(CCOCCOCC(=O)Nc4cccc5c4CN(C4CCC(=O)NC4=O)C5=O)nn3)cc2)cc(-c2ccccc2)c1 ; O=C(O)/C=C/c1cc(C(=O)NCc2ccc(OCc3cn(CCOCCOCC(=O)Nc4cccc5c4CN(C4CCC(=O)NC4=O)C5=O)nn3)cc2)cc(-c2ccccc2)c1 ; "IA"]

Compound 13 was synthesized by coupling the methyl ester derivative of propargyl warhead 8 with Compound 12, prepared as shown in Scheme 2.

methyl (E)-3-(5-((4-((1-(2-(2-(2-((2-(2,6-dioxopiperidin-3-yl)-1-oxoisoindolin-4-yl)amino)-2-oxoethoxy)ethoxy)ethyl)-1H-1,2,3-triazol-4-yl) methoxy)benzyl) carbamoyl)-[1,1 [symbol] biphenyl]-3-yl)acrylate (13): 8 (0.150 g, 0.35 mmol, 1 equiv) and 12 (0.150 g, 0.35 mmol, 1 equiv) were dissolved in a solution of DCM (2.61 mL), MeOH (2.61 mL), and water (1.31 mL). To which CuSO₄·5H₂O (8.8 mg, 0.03 mmol, 0.1 equiv) dissolved in water (6.53 mL) was added and the reaction mixture stirred under a N₂ atmosphere for 5 min at room temperature. Sodium ascorbate (28.5 mg, 0.14 mmol, 0.41 equiv) was dissolved in water (6.53 mL) and added to the reaction mixture. The reaction stirred overnight at room temperature under a N₂ atmosphere. Water was added (13 mL) and the solution was extracted with EtOAc. The organic layer was collected, dried over anhydrous Na₂SO₄, filtered, and concentrated in vacuo. The crude product was purified by flash column chromatography using DCM/MeOH as the eluents to afford the titled compound as a white solid (47.9 mgs, 16%). R_{f}=0.25 (DCM/MeOH=20:1). ¹H NMR (400 MHz, CD₃OD): δ_{H} 1.31 (1H, s), 2.08-2.14 (1H, m), 2.33-2.44 (1H, m), 2.71-2.89 (2H, m), 3.69 (4H, s), 3.81 (d, J=4.0 Hz, 3H), 3.94 (t, J=8.0 Hz, 2H), 4.10 (d, J=4.0 Hz, 2H), 4.44 (d, J=4.0 Hz, 2H), 4.50 (d, J=8.0 Hz, 1H), 4.54 (1H, s), 4.59-4.61 (4H, m), 5.06 (2H, s), 5.09-5.18 (2H, m), 6.69 (d, J=16.0 Hz, 1H), 6.90 (d, J=8.0 Hz, 2H), 7.27 (d, J=8.0 Hz, 2H), 7.39 (t, J=6.0 Hz, 1H), 7.47 (t, J=16.0 Hz, 2H), 7.62-7.73 (4H, m), 7.80 (d, J=16.0 Hz, 1H), 8.00 (1H, s), 8.06 (1H, s), 8.11 (1H, s), 8.17 (1H, s). ¹³C NMR (100 MHz, CD₃OD): δ_{c} 22.7, 30.9, 42.7, 49.8, 50.9, 52.2, 60.9, 68.9, 69.7, 69.9, 70.5, 114.5, 118.9, 120.5, 124.7, 125.0, 126.7, 126.8, 127.3, 127.8, 128.7, 128.7, 129.4, 131.3, 132.2, 132.6, 135.3, 135.4, 135.5, 139.4, 142.3, 143.5, 143.8, 157.4, 167.4, 167.7, 169.5, 169.5, 170.6, 173.2.

(E)-3-(5-((4-((1-(2-(2-(2-((2-(2,6-dioxopiperidin-3-yl)-1-oxoisoindolin-4-yl)amino)-2-oxoethoxy)ethoxy)ethyl)-1H-1,2,3-triazol-4-yl) methoxy)benzyl) carbamoyl)-[1,1 [symbol] biphenyl]-3-yl)acrylic acid (IA): To a stirred solution of 13 (41 mg, 0.05 mmol, 1 equiv) in a mixture of THF/MeOH (1:1) (2 mL) was added aqueous 1N NaOH (5 mg, 0.14 mmol, 3 equiv) solution. The mixture was stirred at 56 °C for 2 hours. The solvent was concentrated in vacuo, and pH was adjusted to 2-5 with 1 N HCl solution. The solution was extracted with EtOAc. The organic layer was collected, dried over anhydrous Na₂SO₄, filtered, and concentrated in vacuo. The precipitate was air-dried to afford the titled compound as a colorless semi-solid (19.3 mgs, 48%). R_{f}=0.13 (Hexane/EtOAc=1:1). ¹H NMR (600 MHz, CD₃OD): δ_{H} 1.31 (2H, s), 2.04 (t, J=9.0 Hz, 1H), 2.17-2.39 (5H, m), 2.84 (1H, s), 3.45 (t, J=9.0 Hz, 1H), 3.70 (d, J=6.0 Hz, 4H), 3.96 (q, J=5.0 Hz, 2H), 4.12-4.13 (2H, m), 4.53-4.56 (3H, m), 4.62 (d, J=4.2 Hz, 2H), 4.95-4.98 (2H, m), 5.08 (2H, s), 6.66 (d, J=18.0 Hz, 1H), 6.92 (dd, J=3.0 Hz, 6.0 Hz, 2H), 7.29 (dd, J=3.0 Hz, 6.0 Hz, 2H), 7.40 (t, J=6.0 Hz, 1H), 7.48-7.50 (3H, m), 7.62 (d, J=6.0 Hz, 1H), 7.72-7.76 (2H, m), 7.79 (d, J=12.0 Hz, 1H). ¹³C NMR (150 MHz, CD₃OD): δ_{c} 15.5, 15.6, 16.7, 24.4, 24.9, 27.9, 28.6, 28.9, 29.5, 29.8, 31.3, 42.3, 45.9, 48.9, 49.4, 53.4, 53.6, 55.4, 55.6, 55.7, 60.4, 68.4, 69.3, 69.5, 70.0, 114.1, 119.5, 119.9, 124.2, 124.5, 126.0, 126.3, 126.3, 126.7, 127.3, 128.1, 128.2, 128.9, 130.8, 131.7, 132.1, 132.2, 134.8, 135.0, 135.1, 139.0, 141.8, 143.1, 143.1, 157.0, 167.3, 168.2, 169.1, 172.6, 174.2, 175.4.

### Biology

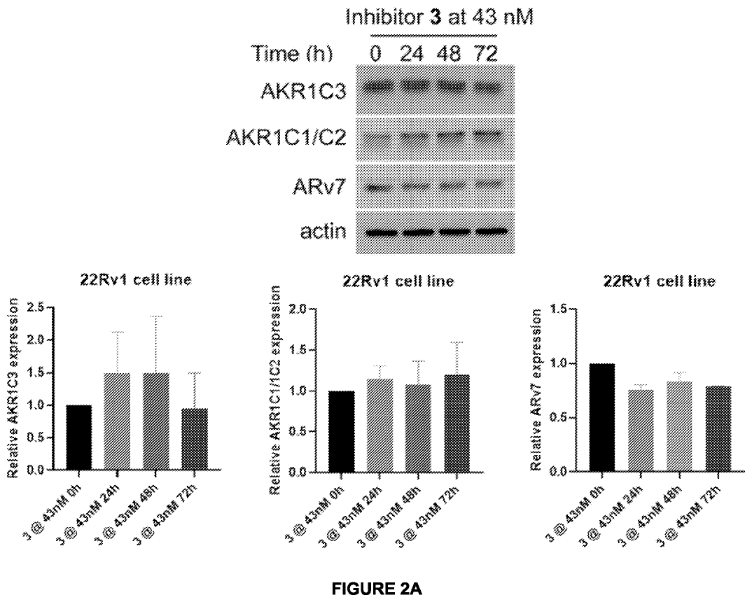

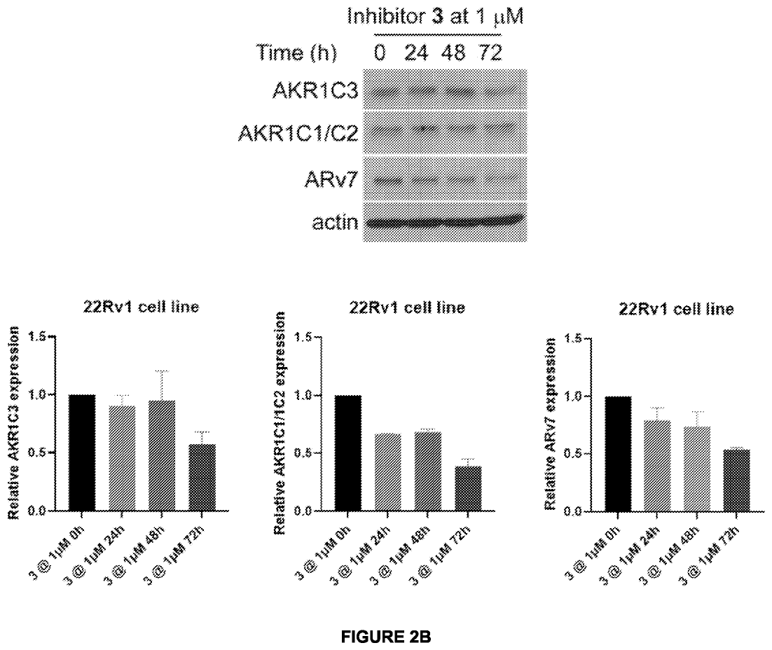

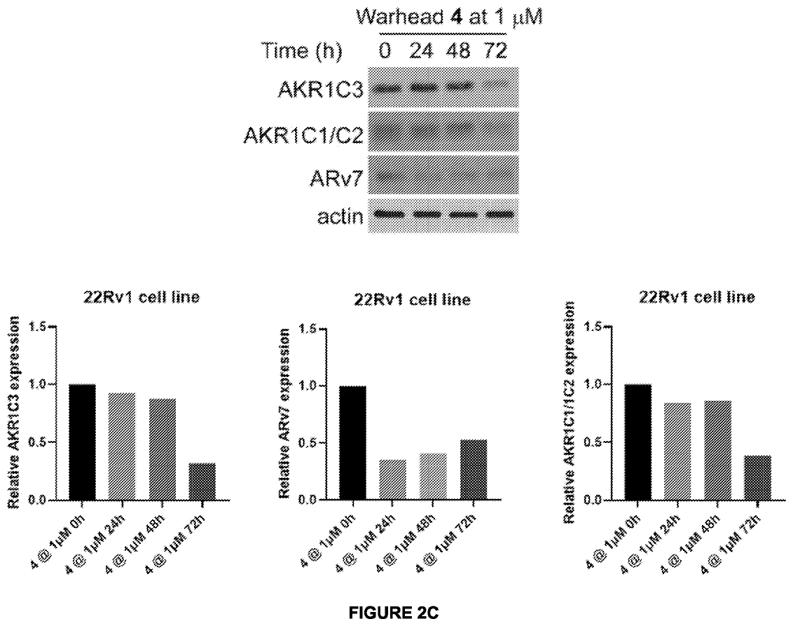

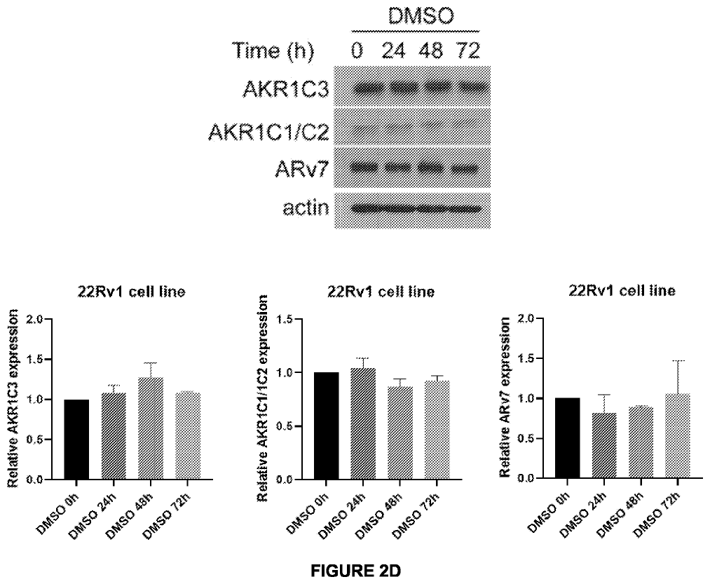

To determine the magnitude of biochemical degradation of ARv7 resulting from AKR1C3 inhibition with a small molecule, the ability of compounds 3 and 4 to degrade AKR1C3, AKR1C1/2 and ARv7 at IC₅₀ and 1 μM concentration were investigated. The results are shown in FIGS. 2A-2D.

Compound 3 at IC₅₀ concentration (43 nM) provided no evidence of degradation of AKR1C3, AKR1C1/2 or ARv7 up to 72 hours (FIG. 2A). At 1 μM concentration, AKR1C3 was degraded by approximately 50% at 72 hours (FIG. 2B) with a time-dependent increase in degradation form 24-72 hours. ARv7 was also degraded in a time-dependent manner with approximately 50% degradation at 72 hours. Interestingly AKR1C1/2 was also degraded in equal quantity (50%) by 3, despite IC50>100 μM versus these two isoforms. Compound 4 at 1 μM proved to be a more efficient degrader at 72 hours than 3. Time-dependent degradation of AKR1C3, AKR1C1/2 and ARv7 were apparent with approximately 70% degradation of all at 72 hours (FIG. 2C). To ensure the observed degradation was the result of AKR1C3 inhibition and not cellular protein variation over time, degradation was monitored over 72 hours following treatment with DMSO (FIG. 2D). Expression levels for the proteins of interest remained constant. Inhibition of AKR1C3 with high concentration of small molecule inhibitor in excess of IC50, leads over 72 hours, to the degradation of AKR1C3 which brings about concomitant degradation of ARv7 and AKR1C1/2, despite these compounds being highly selective for AKR1C3.

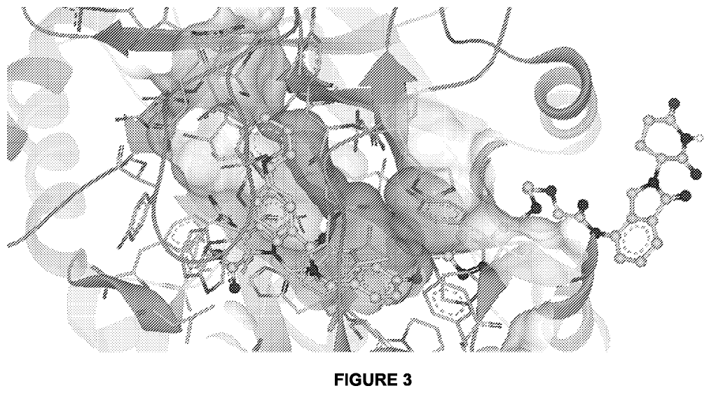

Cognizant that Compound IA is considerably larger than prior AKR1C3 inhibitors of this and other classes, molecular docking studies were conducted to determine fit of the warhead into the SP1 binding site of AKR1C3 and linker length requirement to ensure solvent exposure of the E3 ligase ligand. The results are shown in FIG. 3.

The published crystal structure of AKR1C3 bound with indomethacin (PDB ID: 3UG8) was employed, the ligand was extracted and a binding site of 30 amino acid residues defined. The PROTAC was prepared and docked using SeeSAR 12.1 software (BioSolveIT). Gratifyingly, the docking revealed three promising predictions for the proposed PROTAC design: i) predicted retention of hydrogen bond formation between the warhead and critical amino acid residues Tyr55 and His117 within the AKR1C3 binding site, and are thus predictive of retained AKR1C3 inhibition activity; ii) Nno predicted role for the triazole anchor in binding the AKR1C3 active site, a concern arising from recently disclosed hydroxy triazole AKR1C3 inhibitors; iii) the composition and length of the linker determine the ability of a PROTAC to bind to and stabilize the ternary complex and, as a result, affect polyubiquitination and subsequent degradation of the target protein by the 26S proteasome. In addition, the structure of PROTAC 5 is predicted to engender solvent exposure of the E3 ligase ligand.

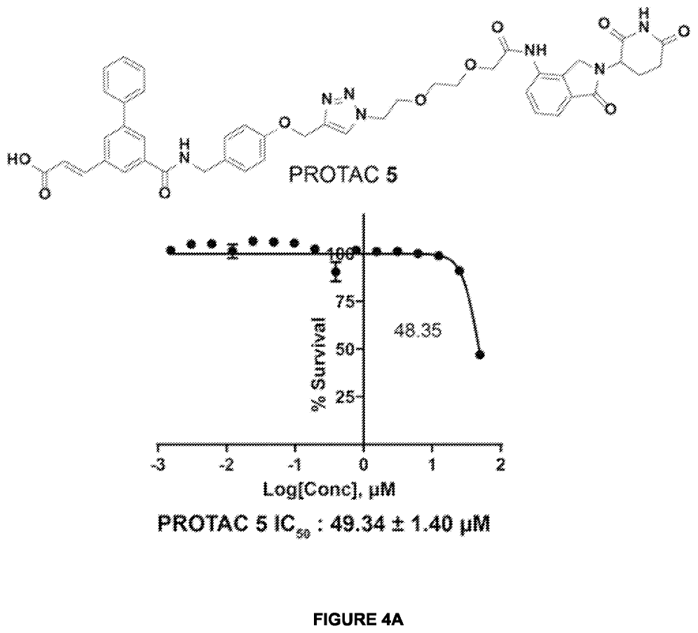

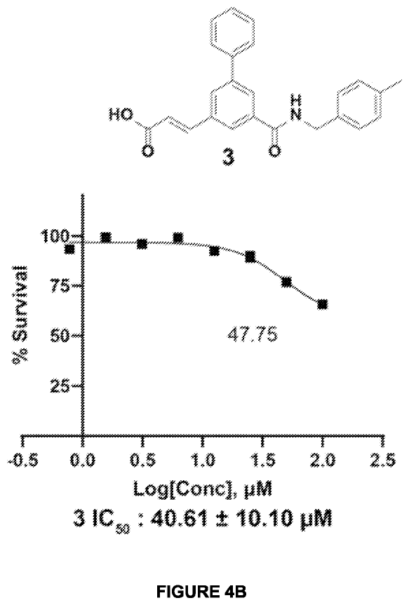

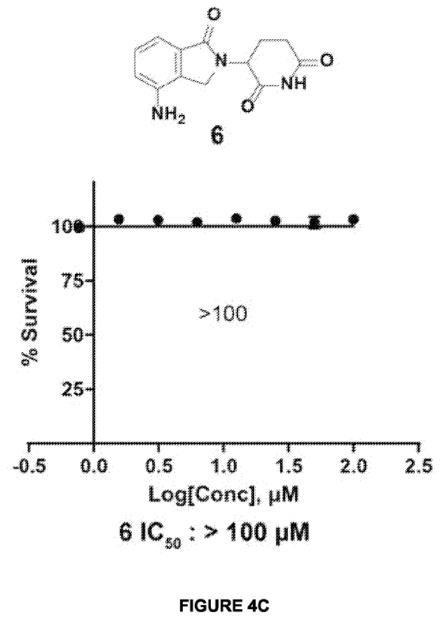

With Compound IA in hand, the biochemical AKR1C3 inhibition activity and ability to ameliorate the survival of 22Rv1 prostate cancer cells that express high levels of AKR1C3 was determined. The results are shown in FIGS. 4A-4C.

Compound IA was found to be equipotent in ameliorating 22Rv1 cell viability to Compound 3 with an IC₅₀=49.34±1.40 μM and 40.61±10.10 μM respectively (FIGS. 4A and 4B). The E3 ligase ligand, lenalidomide 6, a clinical antineoplastic, exhibited no activity in the 22Rv1 prostate cancer cell line (IC₅₀>100 μM), discounting any contribution from this moiety to the activity of Compound IA (FIG. 4C).

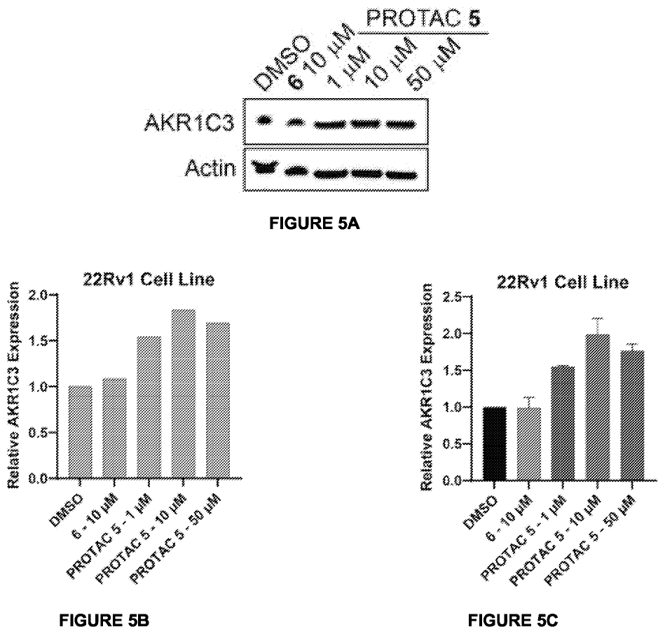

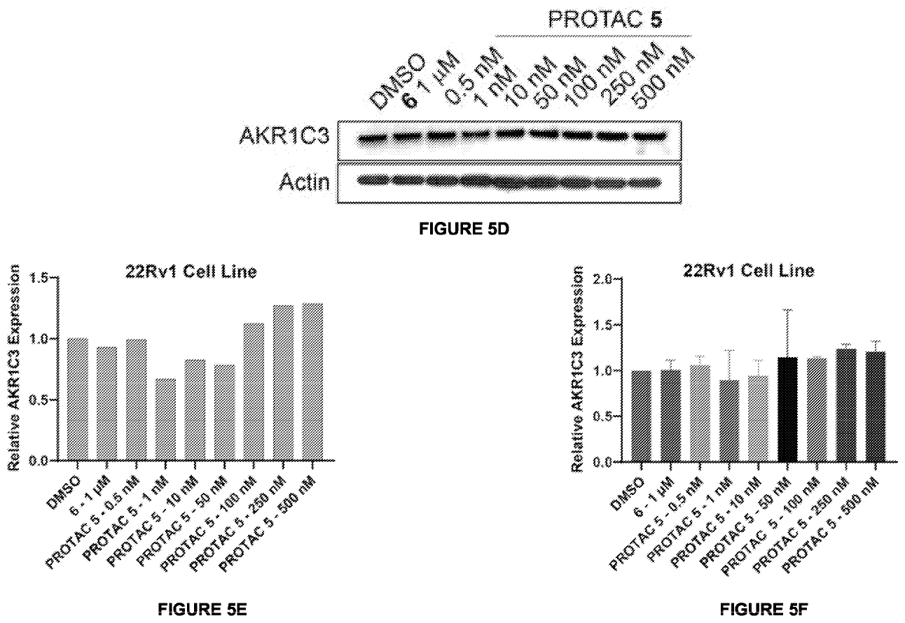

The AKR1C3 degradation effect of PROTAC 5 was screened across a range of concentrations to identify a suitable treatment protocol. 22Rv1 cells were treated with increasing concentrations of Compound IA, DMSO or lenalidomide 6 at 1 μM and 10 μM for 24 hours, and the degradation profile was determined by Western blot analysis with antibody for AKR1C3. The results are shown in FIGS. 5A-5F.

Concentration ranges of 100-500 nM, but particularly 1-50 μM, resulted in increased AKR1C3 expression (FIGS. 5A-C). Without wishing to be bound to any particular theory, it is believed that the higher concentrations can be attributed to the inability of a ternary complex to form, a phenomenon known as the hook effect, where at high intracellular PROTAC concentrations, unproductive binary complexes are favored over ternary complexes, as a result, no degradation occurs. Mean degradation values indicated no degradation at 0.5 nM and very low degradation effect of approximately 10% at 1 and 10 nM (FIG. 5F) which was not statistically significant. Lenalidomide 6 showed no effect on AKR1C3 expression.

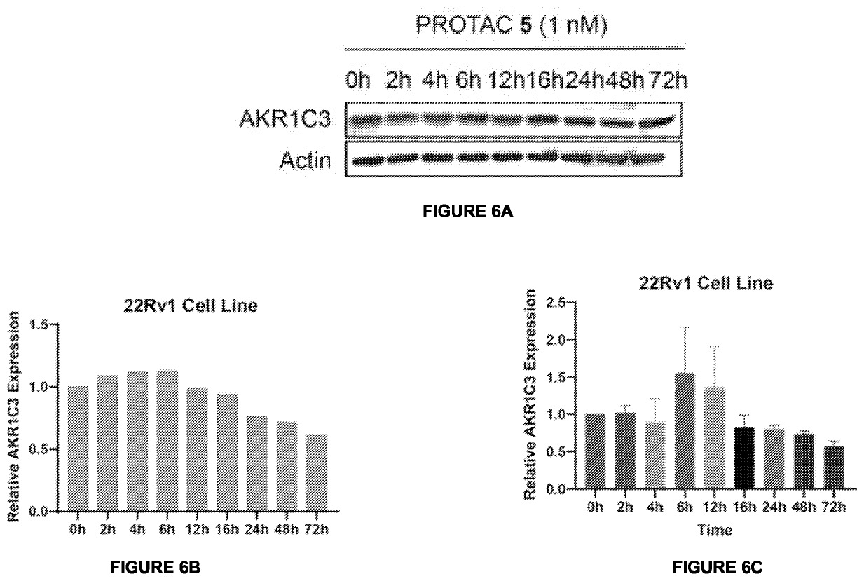

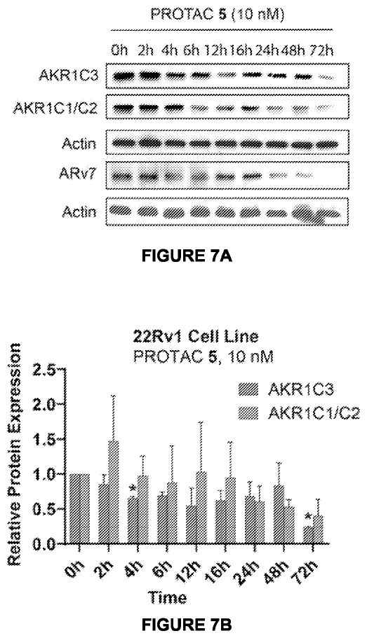

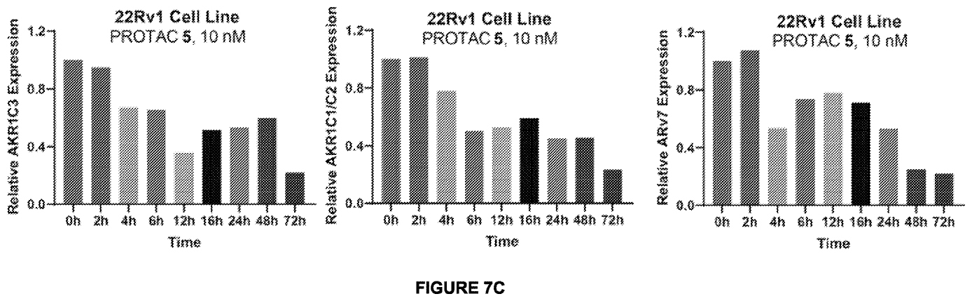

As Compounds 3 and 4 exert their degradation activity at 72 hours (FIG. 2), the time-dependent degradation effect of Compound IA at 0, 2, 4, 6, 12, 16, 24, 48, and 72 h was then determined. The results are shown in FIGS. 6A-6C and FIGS. 7A-7C.

A time-dependent decrease in AKR1C3 levels starting at 24 hours post-treatment, through 72 hours post-treatment was observed with just 1 nM treatment of Compound IA (FIGS. 6A-6C). Gratifyingly, significant degradation of AKR1C3 was observed with 10 nM treatment of Compound IA. Degradation of AKR1C3 was observed from 4 hours post-treatment onwards, with maximal degradation of approximately 75% observed at 72 hours (FIG. 7A-7C). The effect of 10 nM treatment of Compound IA on AKR1C1/C2 degradation was then determined. As seen with Compounds 3 and 4, Compound IA provided a trend of degradation of AKR1C1/C2 in a time-dependent manner (FIG. 7D), but to a lesser extent than AKR1C3. Without wishing to be bound to any particular theory, it is believed that this may be due to the more favorable positioning of a proximal lysine residue for ubiquitination in AKR1C1/C2 that is more hindered in AKR1C3, biological feedback from inhibition of AKR1C3 and/or an as yet to be identified role of AKR1C1/C2 in complex formation and stabilization with ARv7. Furthermore, treatment of 22Rv1 cells with Compound IA resulted in almost complete amelioration of ARv7 expression at 72 hours (FIGS. 7A and 7E).

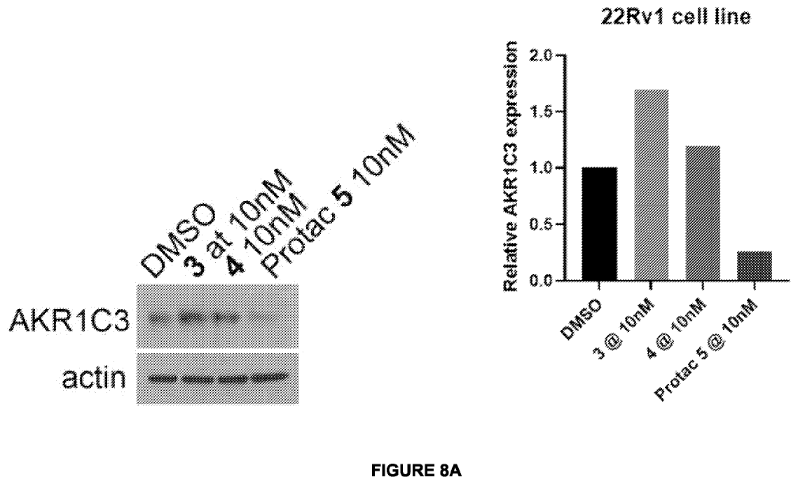

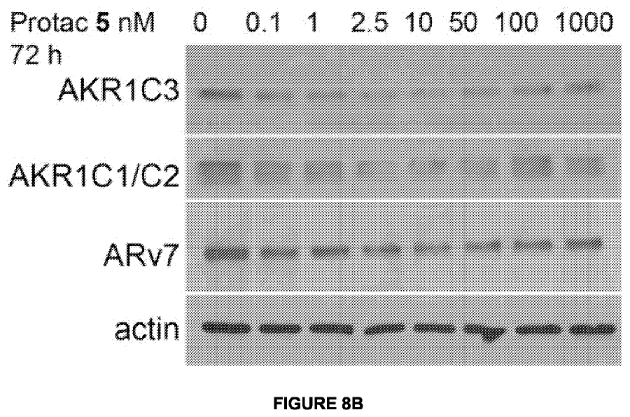

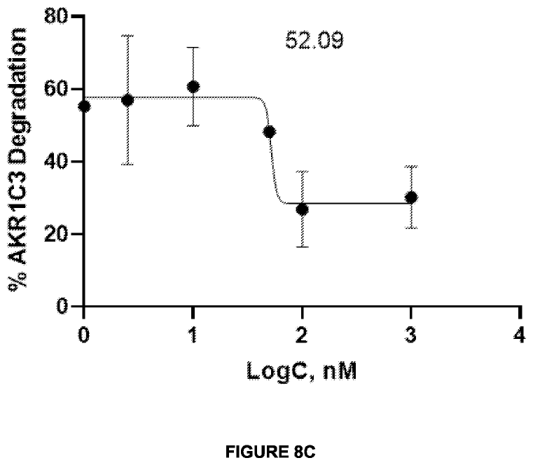

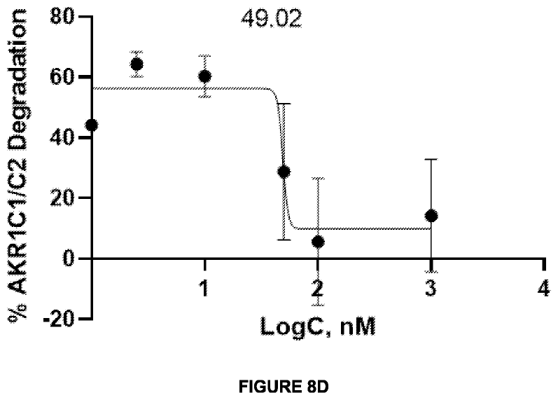

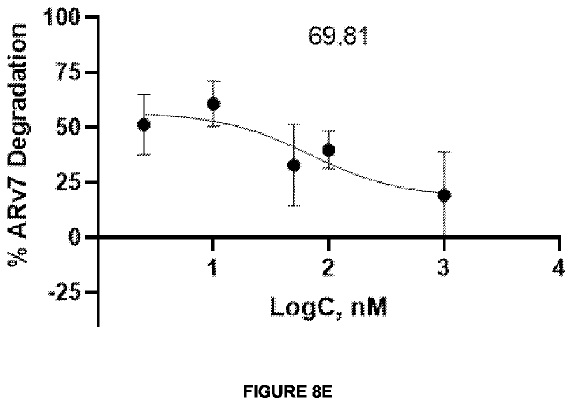

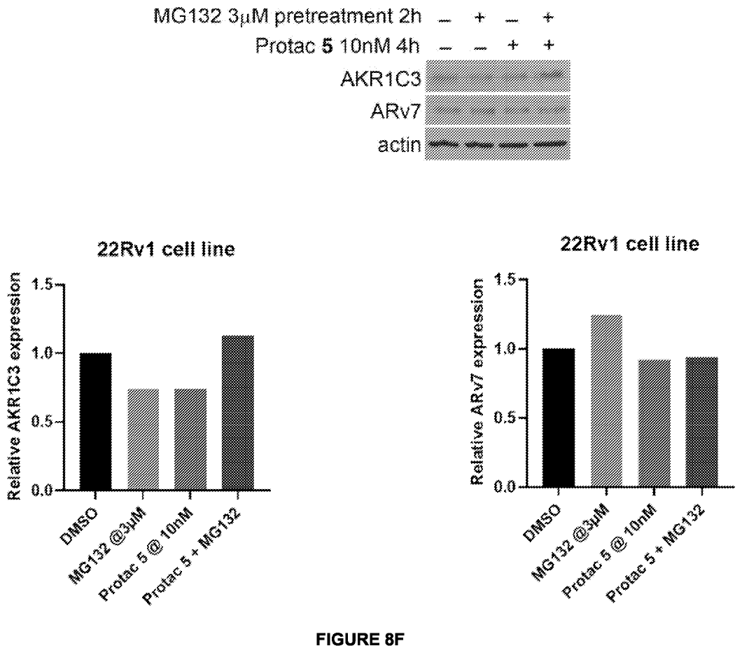

These data demonstrate that compounds of the disclosure (e.g., Compound IA) can provide significant degradation of AKR1C3 expression in 22RV1 prostate cancer cells. Compound IA outperformed Compound 3 at equal 10 nM concentration (FIGS. 8A and 8B) after 72 hour incubation. Compound IA induces 75% degradation of AKR1C3. Calculation of half-maximal degradation concentration (DC₅₀) revealed an AKR1C3 DC₅₀=52 nM, an AKR1C1/C2 DC₅₀=49 nM and an ARv7 DC₅₀=70 nM (FIGS. 8C-8E)

Compound IA at 10 nM effectively reduced AKR1C3 protein expression in 22Rv1 cells with 4 hours of treatment (FIG. 7B). Degradation of AKR1C3 was effectively blocked by pretreatment of the proteasome inhibitor MG132 while ARv7 expression levels were not affected. (FIG. 8F). Without wishing to be bound to any particular theory, these data suggest that Compound IA induces AKR1C3 degradation by a proteasome-dependent mechanism, while ARv7 degradation is brought about as a direct result of AKR1C3 degradation.

In view of the many possible embodiments to which the principles of the disclosure may be applied, it should be recognized that the exemplified embodiments are only examples and should not be taken as limiting the scope of the invention.

### REFERENCES

Adeniji, A. O.; Chen, M.; Penning, T. M. AKR1C3 as a target in castrate resistant prostate cancer. J. Steroid Biochem. Mol. Biol. 2013, 137, 136-149.
Birtwistle, J.; Hayden, R. E.; Khanim, F. L.; Green, R. M.; Pearce, C.; Davies, N. J.; Wake, N.; Schrewe, H.; Ride, J. P.; Chipman, J. K.; Bunce, C. M. The Aldo-Keto Reductase AKR1C3 Contributes to 7,12-Dimethylbenz (a) anthracene-3,4-dihydrodiol Mediated Oxidative DNA Damage in Myeloid Cells: Implications for Leukemogenesis. Mutat. Res. 2009, 662, 67-74.
Stanbrough, M.; Bubley, G. J.; Ross, K.; Golub, T. R.; Rubin, M. A.; Penning, T. M.; Febbo, P. G.; Balk, S. P. Increased Expression of Genes Converting Adrenal Androgens to Testosterone in Androgen-Independent Prostate Cancer. Cancer Research 2006, 66, 2815-2825.
Penning, T. M.; Jonnalagadda, S.; Trippier, P. C.; Rižner, T. L. Aldo-Keto Reductases and Cancer Drug Resistance. Pharmacological Reviews 2021, 73, 1150-1171.
Xiao, X. Z.; Lin, L. Y.; Zhuang, M. K.; Zhong, C. M.; Chen, F. L. Roles of AKR1C3 in malignancy. Chin. Med. J. (Engl.) 2021, 134, 1052-1054.
Liu, Y.; He, S.; Chen, Y.; Liu, Y.; Feng, F.; Liu, W.; Guo, Q.; Zhao, L.; Sun, H. Overview of AKR1C3: Inhibitor Achievements and Disease Insights. J Med Chem 2020, 63, 11305-11329.
Verma, K.; Gupta, N.; Zang, T.; Wangtrakluldee, P.; Srivastava, S. K.; Penning, T. M.; Trippier, P. C. AKR1C3 Inhibitor KV-37 Exhibits Antineoplastic Effects and Potentiates Enzalutamide in Combination Therapy in Prostate Adenocarcinoma Cells. Mol Cancer Ther 2018, 17, 1833-1845.
Bortolozzi, R.; Bresolin, S.; Rampazzo, E.; Paganin, M.; Maule, F.; Mariotto, E.; Boso, D.; Minuzzo, S.; Agnusdei, V.; Viola, G.; the Kronnie, G.; Cazzaniga, G.; Basso, G.; Persano, L. AKR1C enzymes sustain therapy resistance in paediatric T-ALL. British Journal of Cancer 2018, 118, 985-994.
Khanim, F.; Davies, N.; Velica, P.; Hayden, R.; Ride, J.; Pararasa, C.; Chong, M. G.; Gunther, U.; Veerapen, N.; Winn, P.; Farmer, R.; Trivier, E.; Rigoreau, L.; Drayson, M.; Bunce, C. Selective AKR1C3 inhibitors do not recapitulate the anti-leukaemic activities of the pan-AKR1C inhibitor medroxyprogesterone acetate. British Journal of Cancer 2014, 110, 1506-1516.
Verma, K.; Zang, T.; Gupta, N.; Penning, T. M.; Trippier, P. C. Selective AKR1C3 Inhibitors Potentiate Chemotherapeutic Activity in Multiple Acute Myeloid Leukemia (AML) Cell Lines. ACS Med. Chem. Lett. 2016, 7, 774-779.
Verma, K.; Zang, T.; Penning, T. M.; Trippier, P. C. Potent and Highly Selective Aldo-Keto Reductase 1C3 (AKR1C3) Inhibitors Act as Chemotherapeutic Potentiators in Acute Myeloid Leukemia and T-Cell Acute Lymphoblastic Leukemia. J Med Chem 2019, 62, 3590-3616.
Liu, C.; Yang, J. C.; Armstrong, C. M.; Lou, W.; Liu, L.; Qiu, X.; Zou, B.; Lombard, A. P.; D [symbol] bronzo, L. S.; Evans, C. P.; Gao, A. C. AKR1C3 Promotes AR-V7 Protein Stabilization and Confers Resistance to AR-Targeted Therapies in Advanced Prostate Cancer. Mol Cancer Ther 2019, 18, 1875-1886.
Zhang, T.; Karsh, L. I.; Nissenblatt, M. J.; Canfield, S. E. Androgen Receptor Splice Variant, AR-V7, as a Biomarker of Resistance to Androgen Axis-Targeted Therapies in Advanced Prostate Cancer. Clin Genitourin Cancer 2020, 18, 1-10.
Sobhani, N.; Neeli, P. K.; D [symbol] ngelo, A.; Pittacolo, M.; Sirico, M.; Galli, I. C.; Roviello, G.; Nesi, G. AR-V7 in Metastatic Prostate Cancer: A Strategy beyond Redemption. Int J Mol Sci 2021, 22.
Morsy, A.; Trippier, P. C. Reversal of Apalutamide and Darolutamide Aldo-Keto Reductase 1C3-Mediated Resistance by a Small Molecule Inhibitor. ACS Chem. Bio. 2020, 15, 646-650.
Liu, C.; Yang, J. C.; Armstrong, C. M.; Lou, W.; Liu, L.; Qiu, X.; Zou, B.; Lombard, A. P.; D [symbol] bronzo, L. S.; Evans, C. P.; Gao, A. C. AKR1C3 Promotes AR-V7 Protein Stabilization and Confers Resistance to AR-Targeted Therapies in Advanced Prostate Cancer. Molecular Cancer Therapeutics 2019, 18, 1875-1886.
Zhao, J.; Ning, S.; Lou, W.; Yang, J. C.; Armstrong, C. M.; Lombard, A. P.; D [symbol] bronzo, L. S.; Evans, C. P.; Gao, A. C.; Liu, C. Cross-Resistance Among Next-Generation Antiandrogen Drugs Through the AKR1C3/AR-V7 Axis in Advanced Prostate Cancer. Mol. Cancer Ther. 2020, 19, 1708-1718.
Wang, B.; Wu, S.; Fang, Y.; Sun, G.; He, D.; Hsieh, J. T.; Wang, X.; Zeng, H.; Wu, K. The AKR1C3/AR-V7 complex maintains CRPC tumour growth by repressing B4GALT1 expression. J Cell Mol Med 2020, 24, 12032-12043.
Schneekloth Jr., J. S.; Crews, C. M. Chemical Approaches to Controlling Intracellular Protein Degradation. ChemBioChem 2005, 6, 40-46.
Buckley, D. L.; Crews, C. M. Small-Molecule Control of Intracellular Protein Levels through Modulation of the Ubiquitin Proteasome System. Angew. Chem. Int. Ed. 2014, 53, 2312-2330.
Békés, M.; Langley, D. R.; Crews, C. M. PROTAC Targeted Protein Degraders: The Past is Prologue. Nat. Rev. Drug Discov. 2022, 21, 181-200.
Neklesa, T. K.; Winkler, J. D.; Crews, C. M. Targeted Protein Degradation by PROTACs. Pharmacology & Therapeutics 2017, 174, 138-144.
Raina, K.; Crews, C. M. Targeted Protein Knockdown using Small Molecule Degraders. Current Opinion in Chemical Biology 2017, 39, 46-53.
Guo, W. H.; Qi, X.; Yu, X.; Liu, Y.; Chung, C. I.; Bai, F.; Lin, X.; Lu, D.; Wang, L.; Chen, J.; Su, L. H.; Nomie, K. J.; Li, F.; Wang, M. C.; Shu, X.; Onuchic, J. N.; Woyach, J. A.; Wang, M. L.; Wang, J. Enhancing Intracellular Accumulation and Target Engagement of PROTACs with Reversible Covalent Chemistry. Nat. Commun. 2020, 11, 4268.
Lai, A. C.; Crews, C. M. Induced Protein Degradation: An Emerging Drug Discovery Paradigm. Nat. Rev. Drug Discov. 2017, 16, 101-114.
Maddeboina, K.; Jonnalagadda, S. K.; Morsy, A.; Duan, L.; Chhonker, Y. S.; Murry, D. J.; Penning, T. M.; Trippier, P. C. Aldo-Keto Reductase 1C3 Inhibitor Prodrug Improves
Pharmacokinetic Profile and Demonstrates In Vivo Efficacy in a Prostate Cancer Xenograft Model. J Med Chem 2023.
Xiang, W.; Zhao, L.; Han, X.; Qin, C.; Miao, B.; McEachern, D.; Wang, Y.; Metwally, H.; Kirchhoff, P. D.; Wang, L.; Matvekas, A.; He, M.; Wen, B.; Sun, D.; Wang, S. Discovery of ARD-2585 as an Exceptionally Potent and Orally Active PROTAC Degrader of Androgen Receptor for the Treatment of Advanced Prostate Cancer. J Med Chem 2021, 64, 13487-13509.
Pippione, A. C.; Kilic-Kurt, Z.; Kovachka, S.; Sainas, S.; Rolando, B.; Denasio, E.; Pors, K.; Adinolfi, S.; Zonari, D.; Bagnati, R.; Lolli, M. L.; Spyrakis, F.; Oliaro-Bosso, S.; Boschi, D. New aldo-keto reductase 1C3 (AKR1C3) inhibitors based on the hydroxytriazole scaffold. Eur. J. Med. Chem. 2022, 237, 114366.
Zhang, J.; Chen, P.; Zhu, P.; Zheng, P.; Wang, T.; Wang, L.; Xu, C.; Zhou, J.; Zhang, H. Development of Small-molecule BRD4 Degraders Based on Pyrrolopyridone Derivative. Bioorganic Chem. 2020, 99, 103817.
Zang, T.; Verma, K.; Chen, M.; Jin, Y.; Trippier, P. C.; Penning, T. M. Screening baccharin analogs as selective inhibitors against type 5 17beta-hydroxysteroid dehydrogenase (AKR1C3). Chem. Biol. Interact. 2015, 234, 339-348.
Himo, F.; Lovell, T.; Hilgraf, R.; Rostovtsev, V. V.; Noodleman, L.; Sharpless, K. B.; Fokin, V. V. Copper (I)-Catalyzed Synthesis of Azoles. DFT Study Predicts Unprecedented Reactivity and Intermediates. J. Am. Chem. Soc. 2005, 127, 210-216.
Pettersson, M.; Crews, C. M. PROteolysis TArgeting Chimeras (PROTACs)-Past, present and future. Drug Discov. Today Technol. 2019, 31, 15-27.
Robb, C. M.; Contreras, J. I.; Kour, S.; Taylor, M. A.; Abid, M.; Sonawane, Y. A.; Zahid, M.; Murry, D. J.; Natarajan, A.; Rana, S. Chemically induced degradation of CDK9 by a proteolysis targeting chimera (PROTAC). Chem. Commun. (Camb) 2017, 53, 7577-7580.
Wan, Z.; Li, Y.; Bo, S.; Gao, M.; Wang, X.; Zeng, K.; Tao, X.; Li, X.; Yang, Z.; Jiang, Z.-X. Amide bond-containing monodisperse polyethylene glycols beyond 10 000 Da. Org. Biomol. Chem. 2016, 14, 7912.
Zhang, F.; Wu, Z.; Chen, P.; Zhang, J.; Wang, T.; Zhou, J.; Zhang, H. Discovery of a new class of PROTAC BRD4 degraders based on a dihydroquinazolinone derivative and lenalidomide/pomalidomide. Biorg. Med. Chem. 2020, 28, 115228.
Penning, T. M.; Burczynski, M. E.; Jez, J. M.; Hung, C.-F.; Lin, H.-K.; Ma, H.; Moore, M.; Palackal, N.; Ratnam, K. Human 3alpha-hydroxysteroid dehydrogenase isoforms (AKR1C1-AKR1C4) of the aldo-keto reductase superfamily: functional plasticity and tissue distribution reveals roles in the inactivation and formation of male and female sex hormones. Biochem. J. 2000, 351, 67.
Liedtke, A. J.; Adeniji, A. O.; Chen, M.; Byrns, M. C.; Jin, Y.; Christianson, D. W.; Marnett, L. J.; Penning, T. M. Development of potent and selective indomethacin analogues for the inhibition of AKR1C3 (type 5 17β-hydroxysteroid dehydrogenase/prostaglandin F synthase) in castrate-resistant prostate cancer. J. Med. Chem. 2013, 56, 2429.
Rarey, M.; Kramer, B.; Lengauer, T.; Klebe, G. A Fast Flexible Docking Method Using an Incremental Construction Algorithm. J. Mol. Biol. 1996, 261, 470.
Lemmen, C.; Lengauer, T.; Klebe, G. FLEXS: A Method for Fast Flexible Ligand Superposition. J. Med. Chem. 1998, 41, 4502.
Schneider, N.; Lange, G.; Hindle, S.; Klein, R.; Rarey, M. A Consistent Description of HYdrogen Bond and DEhydration Energies in Protein-Ligand Complexes: Methods behind the HYDE Scoring Function. J. Comput. Aided. Mol. Des. 2013, 27, 15.

1. A compound, or pharmaceutically acceptable salt thereof, having the structure of Formula (0):
A-L-B₍₀₎,
wherein
A is a AKR1C3 inhibitor and/or binder moiety;
B is a proteolysis targeting moiety; and
L is a linker moiety.

2.-3. (canceled)

4. The compound or salt of claim 1, wherein the AKR1C3 inhibitor and/or binder moiety is a moiety of formula (A1): 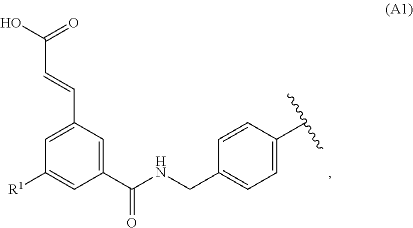
wherein
R¹ is selected from C₁-C₆alkyl, C₆-C₁₀aryl, C₂-C₆alkenyl, C₁-C₆alkoxy, SO₂R⁵, (CO)ₘN(R^{a})₂, and 5-10 membered heteroaromatic having 1-3 ring heteroatoms selected from N, O, S;
R⁸ is C₁-C₆alkyl or substituted or unsubstituted C₆-C₁₀aryl;
each R^{a} independently is H or C₁-C₆alkyl;
m is 0 or 1; and
R¹ is optionally substituted with one or more substituents selected from C₆-C₁₀aryl, 5-15 membered heteroaryl having 1-4 ring heteroatoms selected from N, O, and S, formyl, C₃-C₆cycloalkyl, halo, cycloheteroalkyl-alkylene, hydroxy-C₁-C₆alkylene, methoxymethyl, phenyoxy, cyano, C₁-C₆alkyl, C₁-C₆haloalkyl, and 5-7 membered fused cycloheteroalkyl having 1 to 3 ring heteroatoms selected from N, O, and S.

5. The compound or salt of claim 1, wherein the AKR1C3 inhibitor and/or binder moiety is selected from

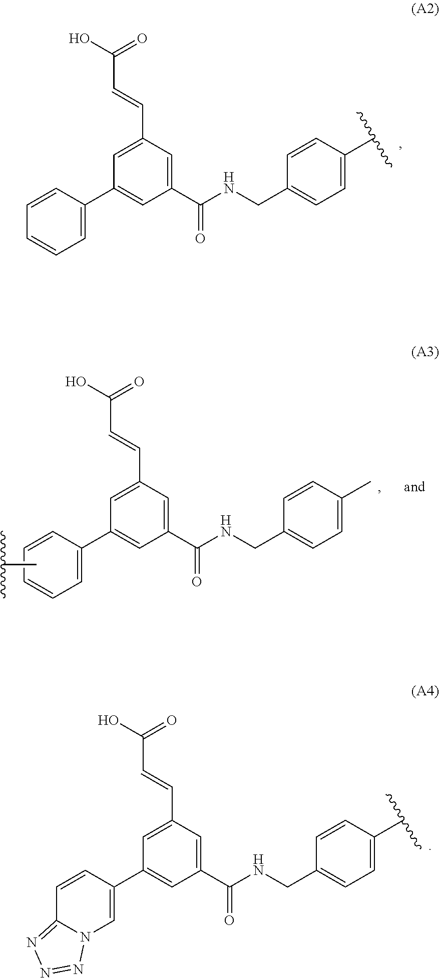

6. The compound or salt of claim 1, wherein the linker moiety is C₁-C₁₈alkylene interrupted with one or more of (i) non-adjacent heteroatom(s) selected from O, S, and NR^{N}, (ii) C₆-C₁₀arylene, (iii) 5-10 membered cycloheteroalkylene having 1-3 ring heteroatoms selected from N, O, and S, and (iv) C(O)NR^{N}, and (v) NR^{N}C(O); and each R^{N} is independently H or C₁₋₆ alkyl.

7. The compound or salt of claim 1, wherein L is selected from 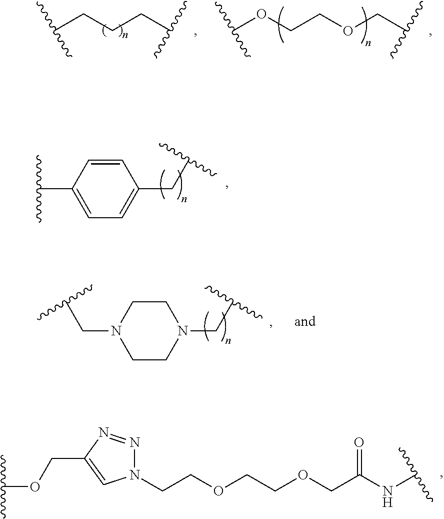
wherein
n is 0-10.

8. The compound or salt of claim 1, wherein the proteolysis targeting moiety is selected from a small molecule moiety, a peptide moiety, an electrophilic moiety, and an antibody moiety or a fragment thereof.

9. The compound or salt of claim 1, wherein the proteolysis targeting moiety is an IκBα-derived moiety, a HIF-1α derived moiety, nutlin, bestatin, methyl-bestatin, thalidomide, pomalidomide, lenalidomide, a thalidomide analog, a VHL binding molecule, an IAP binding molecule, or a terminal alkyne.

10. The compound or salt of claim 1, wherein the proteolysis targeting moiety comprises a E3 ligase binding moiety capable of binding to and/or recruiting an E3 ligase.

11. The compound or salt of claim 10, wherein the E3 ligase binding moiety is VHL, cereblon, MDM2, clAP1, or APC/C^{CDH-1}.

12. The compound or salt of claim 11, wherein the E3 ligase binding moiety is cereblon.

13. The compound or salt of claim 10, wherein the E3 ligase binding moiety has a structure of formula: 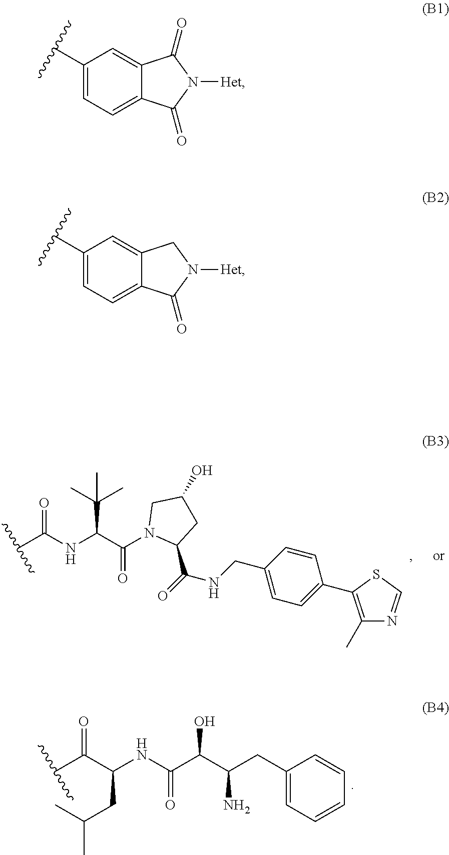
wherein
Het is a 3-7 membered heterocycloalkyl having 1-3 ring heteroatoms selected from O, S, and N, and said heterocycloalkyl is optionally substituted with one or more oxo.

14. The compound or salt of claim 10, wherein the E3 ligase binding moiety has a structure of formula:

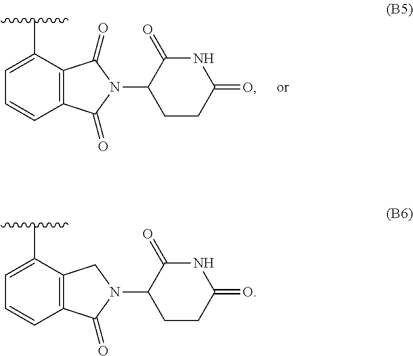 [SMILES: *c1cccc2c1C(=O)N(C1CCC(=O)NC1=O)C2=O ; *c1cccc2c1CN(C1CCC(=O)NC1=O)C2=O]

15. The compound or salt of claim 1, wherein the proteolysis targeting moiety is a terminal alkyne.

16. The compound or salt of claim 4, having the structure of Formula (I) 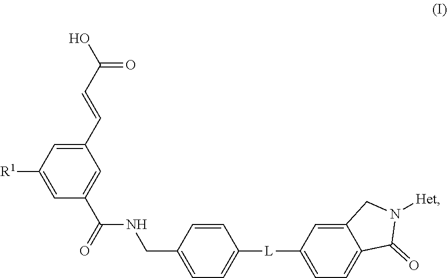
wherein
L is C₁-C₁₈alkylene interrupted with one or more of (i) non-adjacent heteroatom(s) selected from O, S, and NR^{N}, (ii) C₆-C₁₀arylene, (iii) 5-10 membered cycloheteroalkylene having 1-3 ring heteroatoms selected from N, O, and S, and (iv) C(O)NR^{N}, and (v) NR^{N}C(O);
each R^{N} is independently H or C₁-6 alkyl; and
Het is a 3-7 membered heterocycloalkyl having 1-3 ring heteroatoms selected from O, S, and N, and said heterocycloalkyl is optionally substituted with one or more oxo.

17. The compound or salt of claim 16, wherein R¹ is C₆-C₁₀aryl or 6-10 membered heteroaryl having 1-3 ring heteroatoms selected from N, O, or S.

18. The compound or salt of claim 17, wherein R¹ is phenyl or

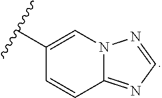 [SMILES: *c1ccc2ncnn2c1]

19. (canceled)

20. The compound or salt of claim 16, having the structure of Formula (IA):

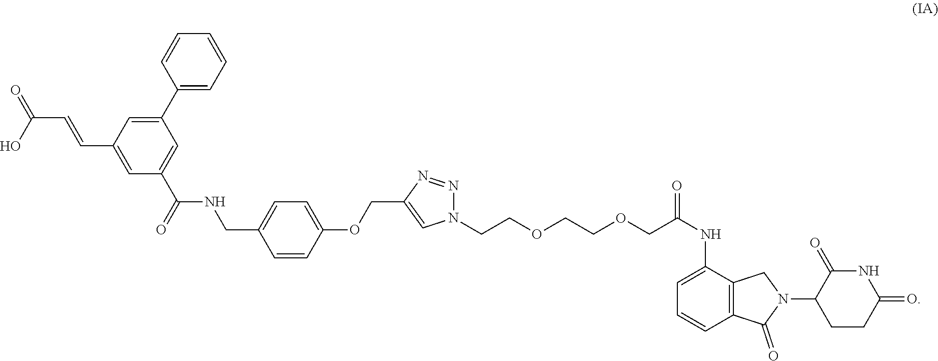 [SMILES: O=C(O)/C=C/c1cc(C(=O)NCc2ccc(OCc3cn(CCOCCOCC(=O)Nc4cccc5c4CN(C4CCC(=O)NC4=O)C5=O)nn3)cc2)cc(-c2ccccc2)c1]

21. The compound or salt of claim 1, having the structure of Formula (II):

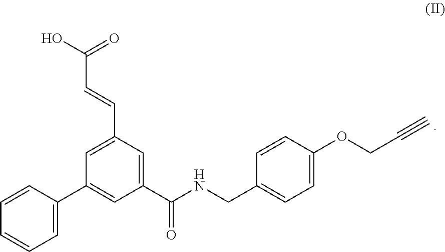 [SMILES: C#CCOc1ccc(CNC(=O)c2cc(/C=C/C(=O)O)cc(-c3ccccc3)c2)cc1]

22. A pharmaceutical composition comprising the compound or salt of claim 1 and a pharmaceutically acceptable carrier or excipient.

23. A method of inhibiting an aldo-keto reductase family 1 member C3 (AKR1C3) comprising contacting the AKR1C3 with a compound of Formula (0): A-L-B, wherein A is a AKR1C3 inhibitor/binder moiety, B is a proteolysis targeting moiety, and L is a linker moiety.

24. The method of claim 23, wherein the proteolysis targeting moiety is selected from a small molecule moiety, a peptide moiety, an electrophilic moiety, and an antibody moiety or a fragment thereof.

25. The method of claim 23, wherein the proteolysis targeting moiety is an IκBα-derived moiety, a HIF-1α derived moiety, nutlin, bestatin, methyl-bestatin, thalidomide, pomalidomide, lenalidomide, a thalidomide analog, a VHL binding molecule, an IAP binding molecule, or a terminal alkyne.

26. The method of claim 23, wherein the proteolysis targeting moiety is a terminal alkyne.

27. A method of degrading an aldo-keto reductase family 1 member C3 (AKR1C3) comprising administering to a subject in need thereof a therapeutically effective amount of a composition comprising a AKR1C3 inhibitor and/or binder moiety linked to an E3 ligase binding moiety, wherein the composition comprises the compound or salt of claim 1.

28. (canceled)

29. A method of treating or preventing a disease or disorder capable of being modulated by AKR1C3 degradation or by androgen receptor splice variant (ARv7), comprising administering to a subject in need thereof a therapeutically effective amount of a composition comprising a AKR1C3 inhibitor moiety linked to an E3 ligase binding moiety.

30. The method of claim 29, wherein the composition comprises the compound or salt of claim 1.

31. The method of claim 29, wherein the disease or disorder is selected from cancer, inflammatory diseases, and neurological diseases.

32. The method of claim 31, wherein the disease or disorder is cancer selected from leukemia, lymphoma, multiple myeloma, breast cancer, prostate cancer, pancreatic cancer, colon cancer, thyroid cancer, bladder cancer, liver cancer, neuroblastoma, brain cancer, lung cancer, ovarian cancer, stomach cancer, skin cancer, cervical cancer, testicular cancer, kidney cancer, carcinoid tumors, and bone cancer, endometriosis, or multiple sclerosis.

33.-53. (canceled)
